(19) United States
(12) Patent Application Publication
(10) Pub. No.: US 2021/0371894 A1
(43) Pub. Date: Dec. 2, 2021

(54) METHOD FOR ANALYZING SAMPLES

(71) Applicant: PATAIGIN, LLC, Baltimore, MD (US)

(72) Inventors: Matthew Sorensen, Baltimore, MD (US); Erik Nilsson, Baltimore, MD (US)

(21) Appl. No.: 17/287,944

(22) PCT Filed: Oct. 25, 2019
(86) PCT No.: PCT/US2019/058213

§ 371 Date: Apr. 22, 2021

Related U.S. Application Data
(60) Provisional application No. 62/750,972, filed on Oct. 26, 2018.
(60) Provisional application No. 62/796,247, filed on Jan. 24, 2019.

(51) Int. Cl. C12Q 1/04; G01N 21/65; H01J 49/16; H01J 49/04; H01J 49/00; G01J 3/44

(52) U.S. Cl. CPC C12Q 1/04; G01N 21/65; H01J 49/164; G01N 2560/00; H01J 49/0031; G01J 3/44; G01N 2201/06113; H01J 49/0418

(57) ABSTRACT

A method for processing samples is presented, said method useful for, among other uses, analysis of components of a sample including lipids, said method further useful for identification of microorganisms, bulk extraction of lipids, detecting infections and other diseases, and other purposes.

### BACKGROUND

The present disclosure relates at least to the fields of analytical chemistry, microbiology, and medicine. More particularly, the present disclosure relates at least to the technical fields of diagnostic medicine and microbial assays.

### BRIEF DESCRIPTION OF THE DRAWINGS

FIG. 1 shows an exemplary plate, according to the present disclosure. FIG. 2 shows an exemplary plate with structured spots, according to the present disclosure. FIG. 3 shows a detailed area of an exemplary plate including a structured spot, according to the present disclosure.

FIG. 4 shows a structured spot and two tubes, according to the present disclosure.

FIG. 5 shows an exemplary assembly comprised of a structured spot and two tubes as shown in FIG. 4, according to the present disclosure.

FIG. 6 shows steps of an exemplary embodiment of a method according to the present disclosure.

FIG. 7 shows an exemplary MALDI mass spectrum of an E. coli sample, according to the present disclosure. FIGS. 8A-8C show exemplary MALDI mass spectra of B. subtilis samples, according to the present disclosure.

FIGS. 9A-9C show exemplary MALDI mass spectra of E. coli samples, according to the present disclosure.

FIG. 10 shows exemplary MALDI mass spectra of various organisms at various concentrations, processed with and without lysozyme, according to the present disclosure.

FIGS. 11A-11C show exemplary ROC curves for classifying species of the Bacillus Cereus Group, based on a method according to the present disclosure.

FIG. 12 shows an exemplary Raman spectrum for a lipid extract of E. coli.

FIGS. 13A and 13B show prespective view of an exemplary enclosure according to the present disclosure.

FIG. 14 shows perspective view of a base of an exemplary enclosure.

FIG. 15 shows an exemplary plate with material dispensed on the plate.

FIG. 16 shows an exemplary plate with two different materials pre-dispensed on the plate.

FIG. 17 shows an exemplary gasket according to the present disclosure.

FIGS. 18A and 18B show an exemplary structured spot according to the present disclosure.

FIGS. 19A-19C show example lipids for three classes of microbes.

FIG. 20 shows an example structure of lipid A.

FIG. 21 depicts an exemplary plate with attached structured spots to which a liquid is dispensed according to the present disclosure.

FIG. 22 depicts an exemplary plate containing wells according to the present disclosure.

FIG. 23 shows exemplary Raman spectra produced according to the present disclosure.

FIGS. 24A-24C show exemplary spectra of gold-tin oxide nanoparticles produced according to the present disclosure. It should be noted that the figures herein are not necessarily to scale.

### SUMMARY

The present disclosure comprises the identification and Antimicrobial Susceptibility Testing (AST) of microorganisms both rapidly and with high sensitivity using extracted membrane lipid profiles. The present disclosure comprises, in at least one aspect, methods for extracting lipids and/or other molecules from a sample.

The present disclosure comprises, in at least one aspect, methods, systems, and kits for identifying or measuring the presence and/or quantity, in a sample, of: one or more microbial species; one or more microbial taxa above the level of species; and/or one or more microbial strains.

The present disclosure comprises, in at least one aspect, methods, systems, and kits for identifying or measuring, in a sample comprising one or more microbial species, one or more microbial taxa above the level of species, and/or one or more microbial strains: an antimicrobial susceptibility or resistance; a virulence; and/or one or more other categories, such as, without limitation, Gram stain.

The present disclosure comprises, in at least one aspect, methods, systems, and kits for identifying or measuring, in a sample comprising one or more microbial species, one or more responses to the environment of a microbe, such as, without limitation, a change in morphology and/or membrane composition in response to exposure to an antimicrobial agent.

The present disclosure comprises, in at least one aspect, methods, systems, and kits for estimating and/or determining the quantity of one or more microbial species, one or more microbial taxa of species, and/or one or more strains in a sample.

The present disclosure comprises, in at least one aspect, methods, systems, and kits for diagnosing a microbial infection or other disease in a subject or a subject sample, and/or making an estimate, a prediction, and/or providing information about the past, present, and/or future status of a disease. Also provided are methods of treating an infection or other disease based on the methods of identifying or measuring, in a sample comprising one or more microbial species.

By way of background, infectious diseases remain a serious health burden throughout the world. Diagnostic tests are critical to treating infectious diseases, but current tests are slow and lack sensitivity. Faster, more sensitive, and/or more accurate tests would allow clinicians to give the right treatment to the patient sooner. Protein fingerprinting tests such as MALDI Biotyper and Vitek-MS have shown that matrix-assisted laser desorption and ionization (MALDI) mass spectrometry can be used for a cost-effective, multiplex microbial test. Herein, a “multiplex” test is a test that simultaneously (i.e., at the same time, or closely apart in time) tests for the individual presence of two or more (e.g., 2, 3, 4, 5, or more) microbial species, strains, taxa, and/or other phenotypes such as antimicrobial resistance. At least one embodiment of the present disclosure comprises a method for extracting molecules including lipids from microbes and for identifying and/or measuring microbes from such extracted molecules including lipids.

Herein, unless specified otherwise or clear from context, “sensitivity” means the ability to identify a microbe from a small number of organisms in a sample. That is, high sensitivity implies a low limit of detection (LOD).

Purposes For Extraction

The presently disclosed embodiments advantageously permit the extraction of molecules, such as lipids, for application to various purposes. In at least one embodiment of the present disclosure, lipids are extracted for one or more of the following purposes.

In at least one embodiment, lipids are extracted suitable for use with a Bacterial Library (BACLIB). One of ordinary skill in the art will appreciate that in one or more embodiments, the methods, systems, and kits of the present disclosure can be used for any of a variety of lipid extraction and analysis tasks. In at least one embodiment of the present disclosure, lipids are extracted from the membranes of microbes for identification and/or quantification of said microbes, and/or for other analysis, including, without limitation, diagnosis, prognosis, and/or determination of antimicrobial resistance of microbes and/or microbial infections.

In at least one embodiment of the present disclosure, lipids are extracted for the purpose of identifying one or more microbe species and/or strains present in a sample. In at least one embodiment of the present disclosure, lipids are extracted for the purpose of identifying one or more microbes in a sample and/or for the purpose of determining the quantity of each and/or the quantity of all of said one or more microbes present in said sample.

By way of background, taxonomic ranks are used for classifying organisms. As used herein, taxonomic ranks are organized as: domain, kingom, phylum, class, order, family, genus, species. Accordingly, it will be understood that, for example, kingdom is a higher taxonomic ranking than domain, and species is a higher taxonomic ranking than genus. In at least one embodiment of the present disclosure, lipids are extracted for the purpose of determining that microbes of one or more taxa are present in a sample, said one or more taxa having a taxonomic rank above species and a taxonomic rank that may be as high or higher than the taxonomic rank of domain, e.g., genus and/or species. In at least one embodiment of the present disclosure, lipids are extracted for the purpose of determining that microbes of one or more categories not corresponding to taxon, such as without limitation Gram stain, are present in a sample.

In at least one embodiment of the present disclosure, lipids are extracted for the purpose of determining an antimicrobial resistance, an antimicrobial resistance potential, and/or an antimicrobial susceptibility of one or more species, taxa, and/or strains of microbes in a sample to an antimicrobial agent, class of agent, combination of antimicrobial agents, and/or combination of classes of antimicrobial agents. In at least one embodiment of the present disclosure, lipids are extracted for the purpose of determining the virulence of a sample. In at least one embodiment of the present disclosure, lipids are extracted for the purpose of determining whether microbes are present above a threshold value comprising one or more of: an absolute number; a concentration per unit volume; a concentration per unit area; a total mass; a mass per unit volume or area a total microbial surface area; a microbial surface area per unit volume or area; a total microbial volume; or a microbial volume per unit volume or area. In at least one embodiment, at least one said threshold concentration corresponds to at least one diagnostic criterion of at least one disease.

In at least one embodiment of the present disclosure, lipids are extracted for the purpose of determining that, below a threshold value of a microbial concentration, number, mass, size, surface area, and/or other microbial measure, one or more microbes of a specific species and/or strain are not present in a sample.

In at least one embodiment of the present disclosure, lipids are extracted for the purpose of determining that, below a threshold value of a microbial concentration, number, mass, size, surface area, and/or other microbial measure, microbes of one or more taxa are not present in a sample, said one or more taxa having a taxonomic rank above species and a taxonomic rank that may be at least as high as the taxonomic rank of domain.

In at least one embodiment of the present disclosure, lipids are extracted for the purpose of determining that, below a threshold value of microbial concentration, number, mass, size, surface area, or other microbial measure, microbes of one or more categories not corresponding to taxa, such as without limitation Gram stain, are not present.

In at least one embodiment of the present disclosure, lipids are extracted for the purpose of refining lipids from a sample or other source for use in a product such as a chemical reagent, chemical marker, affinity reagent or target or component or source of same thereof, drug, drug product, drug ingredient, fuel, or any other product.

Herein, “strain” means any genetic and/or phenotypic variation in an organism below the taxonomic rank of species, including without limitation specific mutations whether previously known or unknown, adaptations to growth conditions, variations related to pathogenicity such as, without limitation, virulence and antibiotic resistance, and/or different life phases of an organism, even if the variation or characteristic has not been typically understood to denote or identify distinct strains. In cases where there is not general agreement on groupings into species, strains, and/or other taxa, “strain” additionally means those organisms that would, in at least one relevant context, be considered to comprise one species, but which are biologically distinct in at least one other relevant context.

### DETAILED DESCRIPTION

The present disclosure relates to methods for processing one or more samples and/or components of samples, to confirm, determine, or estimate information about said samples, and/or to extract chemicals or classes of chemicals of interest from said samples. In at least one embodiment, at least one sample comprises a biological sample. In at least one embodiment, said biological sample contains, may contain, and/or is suspected to contain at least one microbial species. In at least one embodiment, said a least one microbial species comprises one or more of bacteria, archaea, fungi, or protozoa.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means ±20% of the indicated range, value, or structure, unless otherwise indicated. As used herein, the term “about” as applied to mass spectra m/z values means the greater of ±20% or ±100 m/z. As used herein, the term “about” as applied to CFU/mL (colony forming units/mL) means ±10^{0.5}, equivalent to multiplying or dividing the concentration by a factor of approximately 3.16. As used herein, the term “about” as applied to liquid volume means the greater of ±20% or ±0.5 mL. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

The term “consisting essentially of” is not equivalent to “comprising” and refers to the specified materials or steps of a claim, or to those that do not materially affect the basic characteristics of a claimed subject matter.

“Microbial organism,” “microbe,” or “microorganism” as used herein refers to any one of a bacterial species; an archaea; a yeast and/or fungal species; or any microbial species other than bacteria, yeast, or fungi, e.g. protozoa. Herein, “fungus” and “fungi” refer to the kingdom Fungi in its entirety, even for example when fungi are considered in contrast to members of the Fungus kingdom such as yeasts. A microbial organism may exist as a single cell or in a colony of cells. As used herein, “organism” refers to an intact and living organism, and also to an organism or component of an organism that is pre-processed and are or may not be living or comprise a whole or entire organism, such as, without limitation, by fixation in formalin or ethanol. Further, the methods described herein can be applied equally to an organism that is a component or fragment of an organism, so long as the organism that is a component or fragment has, contains, comprises, or is a membrane that is a biological membrane or is a non-biological membrane that contains and/or comprises lipids. For example, the methods described herein can, in certain embodiments, be applied to membrane vesicles including outer membrane vesicles, vesicles of endomycorrhizal fungi, vesicles that are not extracellular vesicles, artificial vesicles, and other types of vesicles.

As used herein, “antimicrobial,” or “a treatment” are used interchangeably to mean any agent used to kill (e.g., a microbicidal agent), inhibit, suppress, or retard the growth of (e.g., a biostatic agent) a microorganism. Antimicrobial agents include, but are not limited to, antibiotics and antifungals.

As used herein, “membrane” includes any membrane on a vesicle, including but not limited to outer membrane vesicles (OMVs), membrane vesicles (MVs), or any membrane fragment, or membrane raft.

In at least one embodiment, a sample is processed, said sample being or comprising one or more of a culture plate colony or smear, a broth culture sample, a blood culture sample, a sample from a biofluid, a clinical sample, or a nonclinical sample. In at least one embodiment, a sample comprises one or more of an environmental sample, a veterinary sample, an agricultural sample, a food or food safety sample, an industrial sample, a process control sample, or a forensic sample. In at least one embodiment, the aforementioned biofluid is a biofluid from a human or non-human source. In at least one embodiment, a sample is processed, said sample comprising or derived from a urine specimen, a blood sample, a sample incubated in a blood bottle, sputum, a sample obtained from sputum, feces, wound effluent, mucus, buccal swab, nasal swab, vaginal swab, nipple aspirate, sweat, saliva, semen or ejaculate, synovial fluid, bronchoalveolar lavage, tears, a urinary catheter sample, a culture plate, or another clinical or medical sample, or another human or mammalian material. In at least one embodiment, a sample comprises a substance or assembly containing at lease one component that is a sample as described herein.

The sample can be used as obtained, or can be processed in any way suitable for use with the methods of this disclosure.

In one embodiment, the methods comprise identifying bacteria directly from a complex sample (i.e., no requirement for amplifying bacteria present in the sample). In another embodiment, bacteria are isolated from the sample, such as by streaking onto solid bacterial culture medium, followed by growth for an appropriate period of time.

The following embodiments and description describe certain embodiments and aspects of the disclosure, without limiting the disclosure in any way. Certain embodiments are numbered and referred to as “EMBODIMENT 1,” etc. Also, certain embodiments provided by way of example for one or more embodiments or the like; such descriptions are not referred to by number.

### Plates

The plate provides, in some embodiments, a surface on which the methods of the present disclosure are practiced.

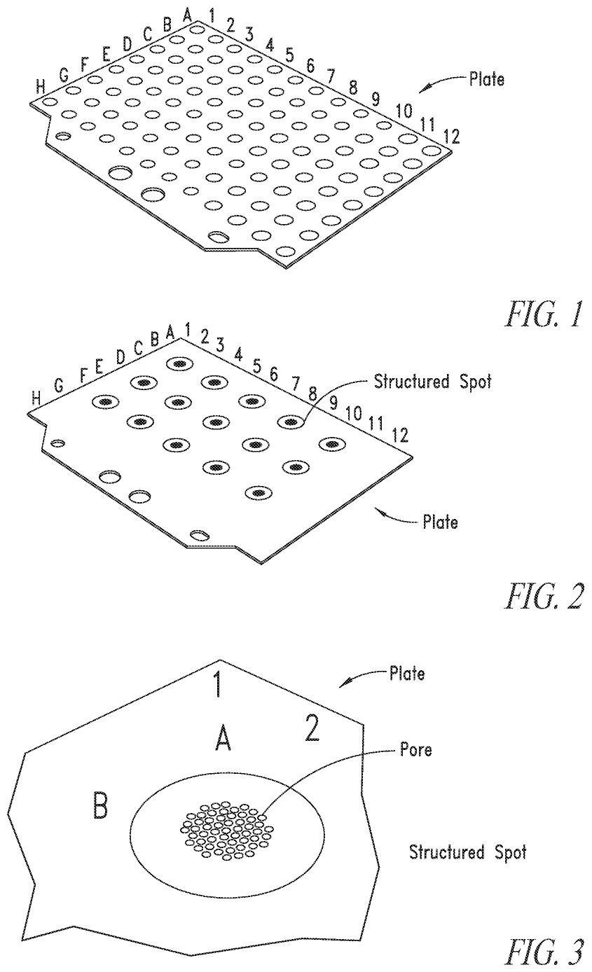

In FIG. 1 is shown a substantially flat object, such a substantially flat object hereinafter referred to as a “plate.” The plate in FIG. 1 has at least one region called a “spot” in or upon which, in at least one embodiment, at least one material is placed. In FIG. 1, spots of the plate are indicated by circles, but in at least one embodiment, at least one spot is indicated by a mark other than a circle (e.g., comprising or consisting of another shape, such as a square, triangle, cross, or the like), or is not indicated by a mark or structure visible to the naked eye.

Certain embodiments described herein relate to operations involving a single sample and/or a single spot. One of ordinary skill in the art will appreciate that a plate may contain more than one spot, such, as without limiting the disclosure, 96 spots or 384 spots. Thus, one of ordinary skill in the art will appreciate that, in at least one embodiment, multiple samples are applied to the one or more spots of a plate. In some embodiments, multiple samples are applied to the same spot of a plate. Furthermore, in at least one embodiment, a single sample is applied to more than one spot of a plate.

Thus, in at least one embodiment, in at least one sequence of steps, a step of applying a sample to a spot or spots is performed once or multiple times to one or more samples and/or one or more spots, whereas at least one other step may comprise applying a sample to a plate as a whole or to a region of a plate as a whole, so that the said at least one other step is not specifically applied per sample or per spot.

One of ordinary skill in the art will appreciate that different embodiments of the present disclosure may be practiced using the same or different spots of one or more plates, while optionally the said one or more plates may have spots which are not used or which can be used for a different application. For example, and without limitation, a sample can be placed on two or more spots, and at least one spot of said two or more spots is used to extract lipids according to a method comprising an embodiment of the present disclosure, while before, after, or in parallel, at least one other spot of said two or more spots can be used for some other purpose (e.g., not for the purpose of extracting the lipid or lipids, or not for the purpose of extracting any lipid, or not for the purpose of extracting from a same sample, or the like). Likewise, one embodiment of the present disclosure can be practiced using at least one spot on a plate, and another embodiment of the present disclosure can practiced using at least one other spot on the plate.

### Plate Constructions

In general, a plate useful in practicing the presently claimed methods, kits, and systems comprises any suitable material for sample analysis, including metals and/or non-metals. Any suitable metal or alloy may be used. In at least one embodiment, a plate comprises steel, such as stainless steel. In some embodiments the stainless steel has been treated by one or more of passivating, pickling, and electropolishing. In at least one embodiment, a plate comprises a metal other than steel. In at least one embodiment, a plate comprises a material that is not a metal.

In at least one embodiment, a plate comprises a composite structure, for example, and without limiting the disclosure, a structure comprising a first layer comprising a first material, and a second layer comprised of or comprising a different material situated on top of the first layer, such that the second layer comprises a surface and has at least one different chemical property as compared to the first layer that affects the shape, composition, and/or movement of liquids on the second layer and/or the first layer. In at least one embodiment, the aforementioned second layer covers only part or portions of the first layer. For example, and without limiting the disclosure, in at least one embodiment, a plate comprises a first layer comprising stainless steel and a second layer comprising a hydrophobic material, wherein the hydrophobic material does not cover some or all of one or more spots on the plate and instead covers some or all of the surface of the plate other than said one or more spots. In some embodiments, the one or more spots are etched or inscribed onto the second layer to visually delineate the areas covered by the hydrophobic second layer outside the spots and the stainless steel first layer within the spots.

As a second example, and without limiting the disclosure, in at least one embodiment, a plate is used that comprises a first layer comprising stainless steel and a second layer comprising a lipophilic material and disposed over at least a portion of the first layer, wherein the lipophilic material covers some or all of at least one spot.

As a third example, and without limiting the disclosure, in at least one embodiment, a plate is used that comprises a first layer comprising stainless steel and a second and a third layer, each of said second and third layers having at least one difference therebetween in a chemical property, and said second and third layers may or may not overlap. In some embodiments the second layer is situated on top of the first layer, and the third layers is situated on top of the second layer. In some embodiments, the third layer covers areas on the first layer not covered by the second layer and so may be situated directly on top of the first layer. In some embodiments the third layer is situated over parts, but not all, of the second layer.

In at least one embodiment, at least one surface of the plate is inscribed, etched, or otherwise modified to include raised or lowered markings or structures (e.g., in a pattern that may extend across all or a portin of the surface), or both raised and lowered markings or structures (e.g., in a pattern that may extend across all or a portin of the surface), such markings or structures providing for identifying one or more visual location to an operator and/or affecting the composition, shape, and/or movement of a liquid on said surface. In at least one embodiment, at least one surface of the plate does not comprise such aforementioned markings or structures.

In at least one embodiment, a plate comprises one or more layers, one or more of which is passivated or otherwise chemically treated or modified. For example and without limiting the disclosure, in at least one embodiment a stainless steel plate comprises two or three layers, each of which has been treated with an acid such as citric or nitric acid to passivate the plate.

### Application of a Sample to a Plate

In some embodiments an end-user will practice the method of the current disclosure of extracting phospholipids and/or other analytes from a sample containing microbial organisms by applying a quantity of liquid from the sample on at least one surface of a plate. In some embodiments, the quantity of liquid, or any substance is placed onto a spot on the plate.

In at least one embodiment, a substance is placed on a spot and rests on or adheres to the spot, so that the spot can be said to “hold” the substance, or also the spot can be said to “contain” the substance, even though the spot may or may not comprise a container in the ordinary sense. Furthermore, the spot can be said to contain the substance, even if there is no definite boundary to the spot, or even if there is at least one definite boundary of the spot but said substance plated on the spot is only partially within said at least one definite boundary.

In at least one embodiment, at least one chemical reagent or other material is applied to at least one spot. For example, and without limiting the disclosure, in at least one embodiment, a mixture comprising citric acid and sodium citrate is applied to at least one spot. As a further example, and without limiting the disclosure, in at least one embodiment, sodium acetate is applied to at least one spot.

As a further example, and without limiting the disclosure, in at least one embodiment, a material that acts as a MALDI matrix is applied to at least one spot. As a further example, and without limiting the disclosure, in at least one embodiment, more than one material is applied to at least one spot. In some embodiments, the MALDI matrix is between about 0.5 μL and 2 μL or about 1 μL of a solution comprising an about 12:6:1 ratio mixture of chloroform:methanol:water to which about 10 mg/mL of beta-carboline has been added. In at least one embodiment, different combinations of materials are applied to two or more different spots. In at least one embodiment, materials are applied to at least one spot but not to at least one other spot. In at least one embodiment, at least one chemical reagent or other material is applied as part of manufacturing the plate. In at least one embodiment, at least one chemical reagent or other material is applied after the plate is manufactured. In at least one embodiment, a kit comprises a plate and one or more reagents, said one or more reagents to be placed on one or more spots by the user of the kit.

In some of the following embodiments, a single sample and/or a single spot is used. In at least one embodiment, a plate has multiple spots. In at least one embodiment, steps below applied to a single spot are performed two or more times to two or more spots of the same plate and/or of different plates, with the same or different samples or specimens used with different spots. In some of the following embodiments, steps applied to a plate as a whole have an effect on some or all of the spots of said plate.

### Bacteria

In various non-limiting embodiments, the methods in the present disclosure can be used to identify one or more bacteria (or sub-species thereof) including but not limited to Escherichia coli, Staphylococcus aureus, Staphylococcus epidermidis, Streptococcus pneumoniae, S. mitis, Streptococcus pyogenes, Stenotrophomonas maltophila, Mycobacterium tuberculosis, Neisseria gonorrhoeae, Neisseria meningitidis, Bordetella pertussis, B. bronchioseptica, Enterococcus faecalis, Salmonella typhimurium, Salmonella choleraesuis, Klebsiella pneumoniae, Pseudomonas aeruginosa, Acinetobacter baumannii, A. calcoaceticus, Bacteroides nordii, B. Salversiae, Enterobacter Subspecies including E. asburiae, E. cloacae, E. hormaechei, E. kobei, E. ludwigii, and E. nimipressuralis, extended spectrum B-lactamase organisms, as well as bacterium in the genus Acinetobacter; Actinomyces, Bacillus, Bacteroides, Bordetella, Borrelia, Brucella, Clostridium, Corynebacterium, Campylobacter, Deinococcus, Escherichia, Enterobacter, Enterococcus, Erwinia, Eubacterium, Flavobacterium, Francisella, Gluconobacter, Helicobacter; Intrasporangium, Janthinobacterium, Klebsiella, Kingella, Legionella, Leptospira, Mycobacterium, Moraxella, Neisseria, Oscillospira, Proteus, Pseudomonas, Providencia, Rickettsia, Salmonella, Staphylococcus, Shigella, Spirillum, Streptococcus, Stenotrophomonas Treponema, Ureaplasma, Vibrio, Wolinella, Wolbachia, Xanthomonas, Yersinia, and Zoogloea.

The various embodiments described herein can be combined to provide further embodiments.

### Embodiment 1: Extraction Of Microbial Lipids

1. Place a sample as described above on a spot of a plate as described above. Optionally allow the sample to dry or partially dry. In at least one embodiment, at least one reagent or other material is pre-applied to at least one spot of the plate before a sample is placed on said at least one spot. The liquid that makes up the sample may have a volume of less than about 0.5 μL, about 0.5 μL, about 1.0 μL, about 1.5 μL, about 2 μL, about 3 μL, about 4 μL, about 6 μL, about 8 μL, about 10 μL, about 20 μL, or greater than 10 μL. The sample may contain microbial organisms at a concentration of greater than 10⁹ Colony Forming Units (CFU)/mL, about 10⁹ CFU/mL, about 10⁸ CFU/mL, about 10⁷ CFU/mL, about 10⁶ CFU/mL, about 10⁵ CFU/mL, about 10⁴ CFU/mL, about 10³ CFU/mL, about 10² CFU/mL, about 10 CFU/mL, about 1 CFU/mL, or less than 1 CFU/mL, or may contains no microbial organisms.

2. Optionally, place at least one reagent or other material on the aforementioned spot, the at least one reagent or other material performing at least one of the following functions: (i) causing, enhancing, or forming part of a chemical reaction in a microbial membrane thereby facilitating material in the microbal membrane to be extracted from the microbial membrane; (ii) causing, enhancing, or forming part of a chemical reaction outside of a microbial membrane to facilitate material extracted from a microbe to be captured or bound on or to at least one surface; (iii) causing, enhancing, or forming part of a chemical reaction outside of a microbial membrane to inhibit material from being bound on or to at least one surface; (iv) acting as a MALDI matrix; (v) acting as a spectroscopy and/or mass spectromtetry standard. For example without limitation, place about 1 μL of a solution of citric acid and sodium citrate on a spot. Optionally allow the sample to dry or partially dry.

3. Heat the plate comprising the spot, sample, and optional reagent or other material. In at least one embodiment, the plate is heated to less than 95° C., about 95° C., about 100° C., about 110° C., about 121° C., about 125° C., about 130° C., about 135° C., or more than 135° C. In at least one embodiment the plate is heated to above 121° C. In at least one embodiment, the plate is heated for less than 15 min., about 15 min, about 20 min, about 25 min, about 30 min, or longer than 30 min.

In an embodiment, a plate is prevented from completely drying while it is heated by heating the plate in a humid atmosphere. In at least one embodiment, the plate is heated in an acidic atmosphere. In at least one embodiment, the plate is heated in an atmosphere containing or comprised of acetic acid vapor. In at least one embodiment, at least one spot of a plate is prevented from partly or completely drying while it is heated by supplying moisture intermittently, periodically, or continuously to the at least one spot on the plate. In some embodiments, the plate is heated in an enclosed space to prevent or minimize evaporation from the surface of the plate.

4. Wash the plate. In at least one embodiment, a plate is washed by at least one application of at least one liquid. In at least one embodiment at least one liquid is applied with a wash bottle. In at least one embodiment, at least one liquid is water. In at least one embodiment, at least one liquid is a composition containing at least one of: water, a detergent, an alcohol, an emulsifier, or an organic solvent, including but not limited to phenol, chloroform, methanol, ethanol, etc. After at least one application of at least one liquid, optionally allow the plate to dry or partially dry.

One skilled in the art will appreciate that, in at least one embodiment, the method of Embodiment 1 extracts molecules that are not lipids in addition to or instead of extracting lipids. Embodiment 1 is also referred to as an exemplary “method for extracting lipids.” As used herein “extraction” and “isolation” are used interchangeably to mean the removal of molecules from a sample, a quantity of liquid, or any substance of interest.

It will be understood that the methods and parameters for extracting lipids may differ for different microbial organisms; for example, extraction from certain organisms may require additional growth time, and different membrane characteristics will affect extraction. Based on this disclosure, it is within the ordinary level of skill in the art to determine appropriate uses and quantities of solvents, detergents, buffers, heating setting, etc. to carry out the methods of the present disclosure.

### Embodiment 2: Mass Spectrometric Analysis

1. Extract lipids according to Embodiment 1.

2. Optionally apply a MALDI matrix to one or more of the spots on which samples have been placed. Allow the plate to dry as necessary.

3. Place the plate in a mass spectrometer or otherwise present some or all of the contents of the one or more spots to a mass spectrometer.

4. Collect mass spectra from the one or more spots. In at least one embodiment, a single spectrum is collected from one or more spots. In at least one embodiment, multiple spectra are collected from one or more spots. In at least one embodiment, multiple spectra with different m/z (mass to charge ratio) ranges are collected from at least one spot. In at least one embodiment, at least one spectrum is collected with a lowest m/z of about less than 100, 100, 300, 400, 600, 700, 800, or 1000 m/z. In at least one embodiment, a spectrum is collected with a lowest m/z of greater than 1000 m/z. In at least one embodiment, a spectrum is collected with a highest m/z of about 800, 1000, 1200, 1500, 1800, 2000, 2200, 2400, or 2500 m/z. In at least one embodiment, a spectrum is collected with a highest m/z of higher than 2500 m/z. In at least one embodiment, two or more spectra are collected that differ in mass spectrometer parameters other than mass range, such as, without limiting the disclosure, detector gain.

In at least one embodiment, a mass spectrometer is used that is an electrospray mass spectrometer, a desorption electrospray ionization (DESI) mass spectrometer, a time-of-flight mass spectrometer, a quadrupole mass spectrometer, a triple quadrupole mass spectrometer, a magnetic sector mass spectrometer, an ion trap mass spectrometer, a quadrupole trap mass spectrometer, an orbitrap mass spectrometer, a gas chromatograph mass spectrometer, a matrix-assisted laser desorption/ionization (MALDI) mass spectrometer, a Time-of-Flight Secondary Ion Mass Spectrometry (TOF-SIMS) mass spectrometer, an ion mobility mass spectrometer, a plasma chromatograph, an inductively-coupled plasma mass spectrometer, a mass cytometer, an accelerator mass spectrometer, a Fourier transform mass spectrometer, a Fourier-transform ion cyclotron resonance mass spectrometer, a mass spectrometer using an ambient ionization method such as direct analysis in real time, a mass spectrometer using a nebulization-ionization method such as surface acoustic wave nebulization, a mass spectrometer using Rapid Evaporative Ionization Mass Spectrometry, or another type of mass spectrometer.

### Embodiment 3: Spectroscopic Analysis

1. Extract lipids according to Embodiment 1.

2. Analyze the plate with a spectroscopic instrument, where said instrument operates by laser induced fluorescence spectroscopy, atomic absorption spectroscopy, atomic emission spectroscopy, flame emission spectroscopy, acoustic resonance spectroscopy, cavity ring down spectroscopy, circular dichroism spectroscopy, Raman spectroscopy, surface enhanced Raman spectroscopy, coherent Raman spectroscopy, cold vapor atomic fluorescence spectroscopy, nuclear magnetic resonance spectroscopy, electrical impedance spectroscopy, electron phenomenological spectroscopy, electron paramagnetic resonance spectroscopy, Fourier-transform spectroscopy, laser-induced breakdown spectroscopy, photoacoustic spectroscopy, photoemission spectroscopy, photothermal spectroscopy, spectrophotometry, vibrational circular dichroism spectroscopy, gamma spectroscopy, flow cytometry, or some other type of spectroscopy; or by means of a scintillation detector, scintillation counter, Geiger counter, ionization chamber, gaseous ionization detector, or other radiation detector.

### Embodiment 4: Combined Analysis

Collect both mass spectrometric (Embodiment 2) and spectroscopic (Embodiment 3) information on a sample, by performing at least one of the following steps.

1. Perform the steps of both Embodiment 2 and Embodiment 3. In at least one embodiment, Embodiment 2 is performed before Embodiment 3, Embodiment 3 is performed before Embodiment 2, or Embodiments 2 and 3 are performed in parallel. In at least one embodiment, Embodiment 2 and Embodiment 3 are performed on one or more spots on the same plate. In at least one embodiment, at least two spots on the same or on different plates are used for performing at least one of Embodiment 2 or Embodiment 3.

2. Perform the steps of Embodiment 2, then using the same plate, perform the steps of Embodiment 3, beginning with step 2 of Embodiment 3.

3. Perform the steps of Embodiment 3, then using the same plate, perform the steps of Embodiment 2, beginning with step 2 of Embodiment 2.

### Embodiment 5: Classification Of Spectra

1. Optionally preprocess at least one set of spectroscopic information. For example, and without limiting the disclosure, in at least one embodiment, at least one set of spectroscopic information is preprocessed by at least one of baseline correction, alignment, charge state deconvolution, isotopic deconvolution, Fourier transform, a type of integral transform other than Fourier transform, peak extraction, or another kind of feature extraction. In at least one embodiment, a sequence of preprocessing operations is performed on one or more spectra and at least one additional sequence of preprocessing operations is performed on the one or more spectra and/or on one or more additional spectra, where each the aforementioned spectra is obtained from a distinct sample or alternatively at least two of the aforementioned spectra are obtained from the same sample.

2. Classify at least one spectrum using a classification method.

For example, and without limiting the disclosure, in at least one embodiment at least one spectrum is classified by comparing the spectrum to an exemplar, average, consensus, or synthetically generated spectrum from a library. As a further example, in at least one embodiment, at least one spectrum is classified partly or entirely without comparison to another spectrum. For example, and without limiting the disclosure, in at least one embodiment, at least one spectrum is classified using a machine learning method such as by using a support vector machine. One skilled in the art will appreciate that certain machine learning methods are typically performed using machine learning models that have previously been trained on training data with properties related to the data to be classified.

In at least one embodiment, classification is performed on a single spectrum or on features extracted from a single spectrum from at least one sample. In at least one embodiment, classification is performed on at least two spectra or on features extracted from at least two spectra. In at least one embodiment, the aforementioned at least two spectra are from the same spot on the same plate. In at least one embodiment, a first spectrum of at least two spectra is from a first spot on a first plate whereas a second spectrum of the at least two spectra is from a second spot on the first plate or on a second plate. In at least one embodiment, three or more spectra from one or more spots are used. In at least one embodiment, at least two spots are processed under essentially the same conditions. In at least one embodiment at least two spots are processed differently, using any or all of at least the various methods discussed in the following embodiments, including without limitation use of buffers with different compositions and/or in different amounts, or use of lysozyme in different amounts.

In at least one embodiment, at least one spectrum used for training data and/or as an exemplar, average, or consensus spectrum is produced by a method described in one of the embodiments or elsewhere herein. In at least one embodiment, at least one spectrum used for training data and/or exemplar, average, or consensus spectra is produced by some other method besides a method described in one of the embodiments or elsewhere herein.

### Embodiment 6: Classification of Samples

1. Obtain at least one spectrum from at least one sample by performing the steps of Embodiments 2, 3, or 4.

2. Classify samples by performing the steps of Embodiment 5 one or more times on the at least one spectrum obtained in step 1.

In at least one embodiment, at least one sample is classified hierarchically. The at least one sample is first classified at a more general level by performing the steps of Embodiment 5 for a first classification, then the at least one sample is classified to a more specific level by performing the steps of Embodiment 5 again for a second classification; wherein either the same spectrum is used for the aforementioned first classification and second classification or else at least one spectrum is used for the first classification and not the second classification, and/or at least one spectrum is used for the second classification and not the first classification. Furthermore, in at least one embodiment, the aforementioned hierarchical classification is extended in like manner to a third classification step or to any number of classification steps.

### Embodiment 7: Screen Samples

1. According to the steps of Embodiment 6, using at least one spectrum, classify at least one sample for the presence of microbes or for the presence of microbes above a threshold amount, or conversely classify at least one sample for the absence of microbes or for the absence of microbes below a threshold amount, or classify at least one sample for the presence or absence or for the presence or absence above or below a threshold amount of a taxon of microbe such as without limitation bacteria, or for the presence or absence or for the presence or absence above or below a threshold amount of one or more categories not corresponding to taxon, such as without limitation Gram stain. In an embodiment, at least one spectrum is a Raman spectrum.

2. If in step 1 the aforementioned sample is determined to have microbes of interest present or present above a threshold value, then according to the steps of Embodiment 6, optionally using different spectra from step 1, classify the sample according to at least one of microbial species, strain, taxon above the level of species, antimicrobial susceptibility or resistance, virulence, and/or one or more other categories. In an embodiment, step 2 is performed using at least one additional spectrum, and the at least one additional spectrum is a MALDI spectrum.

For example without limitation, in an alternative embodiment, a Raman spectrum of a sample determines the presence or absence of microbes belonging to one or more classes in the sample, said one or more classes either corresponding to taxa or not corresponding to taxa, then a MALDI mass spectrum determines the identity of microbes present at a greater refinement than was determined with the Raman spectrum, such as without limitation microbial species, strain, taxon above the level of species, antimicrobial susceptibility or resistance, or virulence.

### Embodiment 8: Classify Samples Via Mass Spectrometry

According to the steps of Embodiment 6, using at least one mass spectrum, classify a sample according to at least one of microbial species, strain, taxon above the level of species, strain, antimicrobial susceptibility or resistance, virulence, and/or one or more other categories.

### Embodiment 9: Classify Samples Via Maldi

According to the steps of Embodiment 6, using at least one MALDI mass spectrum, classify a sample according to at least one of microbial species, strain, taxon above the level of species, strain, antimicrobial susceptibility or resistance, virulence, and/or one or more other categories.

### Embodiment 10: Classify Samples Via Spectroscopy

According to the steps of Embodiment 6, using at least one spectrographic spectrum, classify a sample according to at least one of microbial species, strain, taxon above the level of species, strain, antimicrobial susceptibility or resistance, virulence, and/or one or more other categories.

### Embodiment 11: Classify Samples Via Raman Spectroscopy

According to the steps of Embodiment 6, using at least one Raman spectrum, classify a sample according to at least one of microbial species, strain, taxon above the level of species, strain, antimicrobial susceptibility or resistance, virulence, and/or one or more other categories.

### Embodiment 12: Structured Spot

Perform any of Embodiments 1-11 or 13-33, using a plate with at least one structured spot.

FIG. 2 depicts a plate with structured spots. FIG. 3 depicts a detailed area of a plate with one or more structured spots.

In at least one embodiment, at least one structured spot consists primarily or entirely of a substantially flat object or surface. In at least one embodiment, at least one structured spot has pores or holes penetrating it. In at least one embodiment, at least one structured spot does not have holes or pores, and/or has depressions or channels that are open at only one end. In at least one embodiment, at least one structured spot contains pores that are are roughly circular. In at least one embodiment, at least one structured spot contains pores that are smaller than about 2 μm in diameter. In at least one embodiment, at least one structured spot contains pores that are larger than about 10 nm in diameter. In at least one embodiment, at least one structured spot contains pores that are between about 100 nm and about 1 μm in diameter.

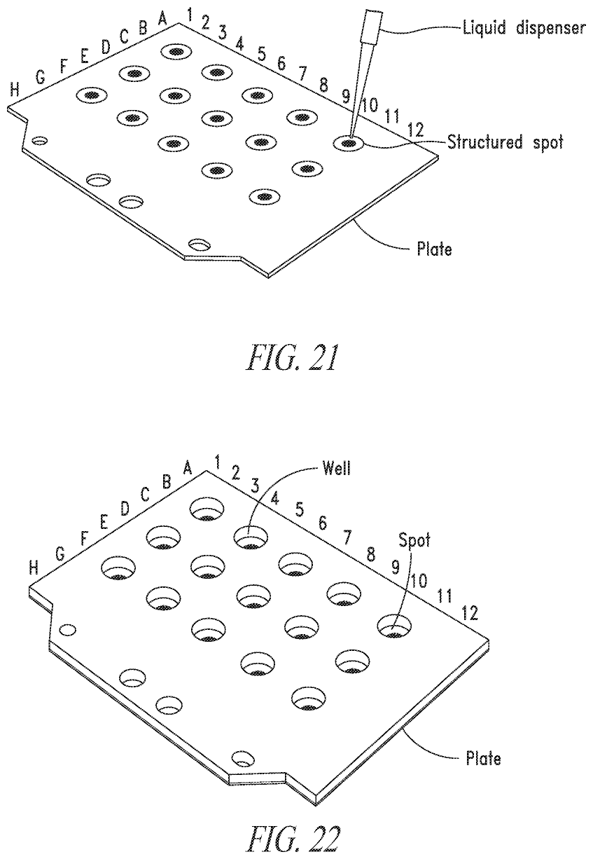

In at least one embodiment, at least one structured spot contains or is situated in a well or cavity. For example without limitation, in at least one embodiment, a structured spot is situated on the bottom of and/or comprises the bottom surface of a well such as a microtiter-type plate. In at least one embodiment, at least one structured spot comprises part of a microtiter plate such as is shown in FIG. 22.

In at least one embodiment, at least one structured spot comprises in part at least one metamaterial and/or microstructure, such as without limitation a particle or surface composed of or containing one or more of carbon, a carbon nanotube, graphene, graphene oxide, a carbon nanocomposite, tungsten oxide, a gold-plated anodized aluminum oxide thin film, silicon, a nanoparticle, a nanowire, gold, silver, copper, titanium, a particle composed of a metal, gold-tin composite, nickel oxide, titanium oxide, zinc oxide, platinum, gold-platinum nickel oxide, zinc oxide-titanium oxide, a magnetic particle, magnetite, cadmium sulfide, a carbon particle, selenium, cadmium telluride, rhenium oxide, silica, porous silicon, tin, tin oxide, platinum nanosponge, nanosponge made of a material other than platinum, a metal-organic framework, a metal-organic framework nanosheet, aluminum-doped zinc oxide, boron doped nano-diamond, or proton sponge.

In at least one embodiment, at least one structured spot comprises in whole or in part at least one metamaterial such as without limitation one or more composite nanoparticles containing at least one of a metal, a metal oxide, a metal sulfide, or any of the materials mentioned elsewhere in Embodiment 12.

One skilled in the art sometimes refers to a surface with pores such as in a structured spot as a “membrane” or “filter.” Sometimes, one skilled in the art refers to an entire object such as at least one instance of a structured spot, an object containing a structured spot, or an object comprising part of a structured spot as a “membrane,” “support,” or “filter.” Such use of the term “membrane” is distinct from a biological membrane, such as found in a cell wall. In at least one embodiment, at least one structured spot performs the function of at least one of a filter, support, or membrane. In at least one embodiment, at least one structured spot does not performs the function of at least one of a filter, support, or membrane.

In at least one embodiment, at least one structured spot comprises in whole or in part a surface with affinity for at least one microbial lipid and/or with affinity for at least one microbial membrane. For example without limitation, in at least one embodiment, at least one structured spot comprises in whole or in part a surface and/or surface coating that is stainless steel, a metal other than stainless steel, a metal oxide, lysozyme, limulus amebocyte lysate, indium tin oxide, an antibiotic, an antifungal agent, an immobilized antibiotic or antifungal agent, an amphotericin or an immobilized amphotericin, or eosinophil cationic protein. In at least one embodiment, at least one surface or other structure performs at least one other function in addition to or instead of affinity, such as without limitation as a MALDI or MALDI-like matrix, as an internal standard, as a catalyst, or as a promoter of lipid extraction and/or membrane destabilization by means other than catalysis. In at least one embodiment at least one surface as described herein is the surface of a particle and/or a nanoparticle. In at least one embodiment at least one surface as described herein contains, has embedded in it, and/or has placed on it a particle and/or a nanoparticle.

In at least one embodiment, one or more structured spots are integral parts of the aforementioned plate. In at least one embodiment, one or more structured spots are constructed separately from the plate then attached to the plate. In at least one embodiment, at least one plate is distributed with at least one structured spot attached to said plate. In at least one embodiment, one or more structured spots are distributed separately from at least one plate, then attached to at least one plate after distribution. In at least one embodiment, at least one structured spot that is attached to a plate is removed after use then replaced by at least one replacement structured spot.

In at least one embodiment, at least one structured spot is made in whole or in part of alumina. In at least one embodiment, at least one structured spot comprises in whole or in part alumina self-assembled by electrolysis. In at least one embodiment, at least one structured spot comprises in whole or in part alumina self-assembled by electrolysis on a metal substrate, said substrate later removed by chemical etching, leaving an alumina structure with pores.

In at least one embodiment, at least one thin layer is added to one or more surfaces of a structured spot. In at least one embodiment, at least one structured spot comprises alumina plated in gold, silver, a platinum group metal, or some other metal.

In at least one embodiment, at least one structured spot and/or at least one component of at least one structured spot has at least one of the following functions:

(i) As a MALDI matrix or matrix-like material, either as the only matrix or in addition to or combination with another matrix material.

(ii) As a filter, allowing liquid and small objects to pass through a mesh or pores, while larger objects are retained on at least one surface of the structured spot. For example without limiting the disclosure, in at least one embodiment a liquid biological sample is filtered with a structured spot. As a further example, in at least one embodiment, a urine sample is filtered with a structured spot.

In at least one embodiment, at least one sample is filtered to concentrate microbes on at least one spot from a liquid sample. In at least one embodiment, at least one sample is filtered to separate relatively large objects from small objects in the at least one sample. In at least one embodiment, at least one sample is filtered to desalt the sample, or to remove or reduce the amount or concentration of some chemical other than a salt or ions associated with a salt. In at least one embodiment, at least one sample is filtered to allow wash water from a washing step to be removed from the surface of the plate that is not the surface of said plate being washed.

In at least one embodiment, filtering is accomplished with at least one structured spot attached to a plate. For example without limiting the disclosure, a volume of liquid is repeatedly or continuously dispensed onto one side of a structured spot. The filtered liquid flows through pores in the structured spot, where it can accumulate or fall off the other side of the structured spot and/or the plate. In at least one further embodiment, objects larger than the pore size such as microbes on the said one side are retained on the said one side of the spot.

(iii) As an enhanced surface for Surface-enhanced Raman spectroscopy.

(iv) As a structured droplet attachment, causing materials dissolved or suspended in the spot to be concentrated in a smaller area. In an embodiment, at least one structured spot on a relatively hydrophobic surface prevents or reduces the “coffee ring effect.”

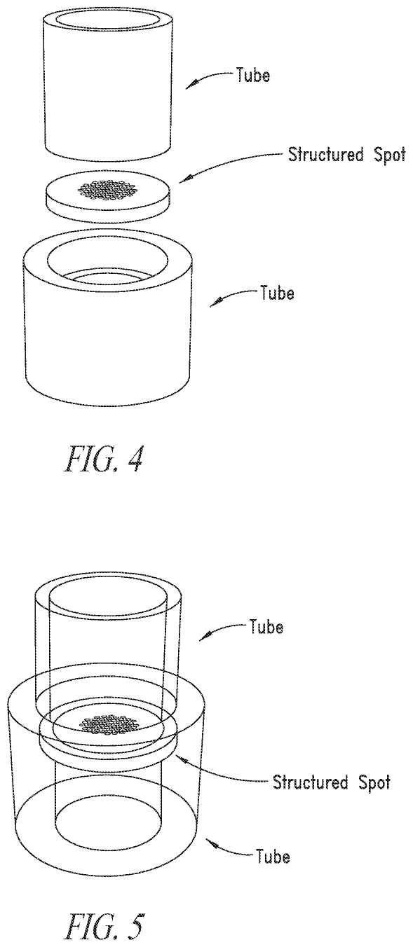

FIG. 4 depicts a structured spot and two tubes. FIG. 5 depicts an assembly of a structured spot and two tubes. In at least one embodiment, liquid is applied into one of the two tubes, passing through the pores of the structured spot. Filtered liquid exits the other of the two tubes, and objects such as microbes larger than the pore size are retained on the structured spot. In at least one embodiment, the assembly in FIG. 5 is used to filter a liquid sample, then the structured spot is removed from the assembly and attached to a plate.

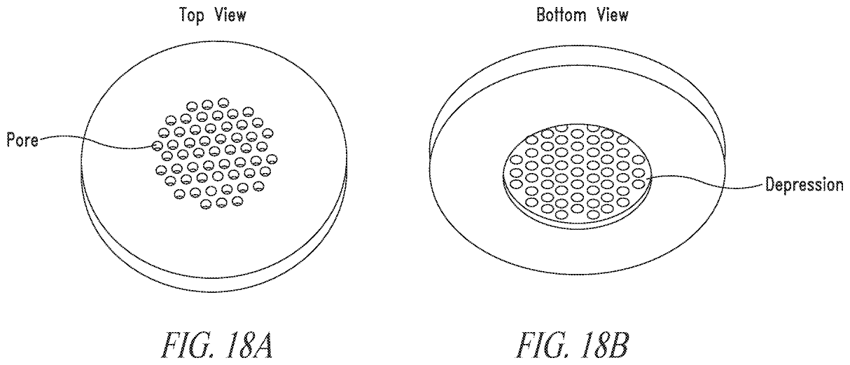

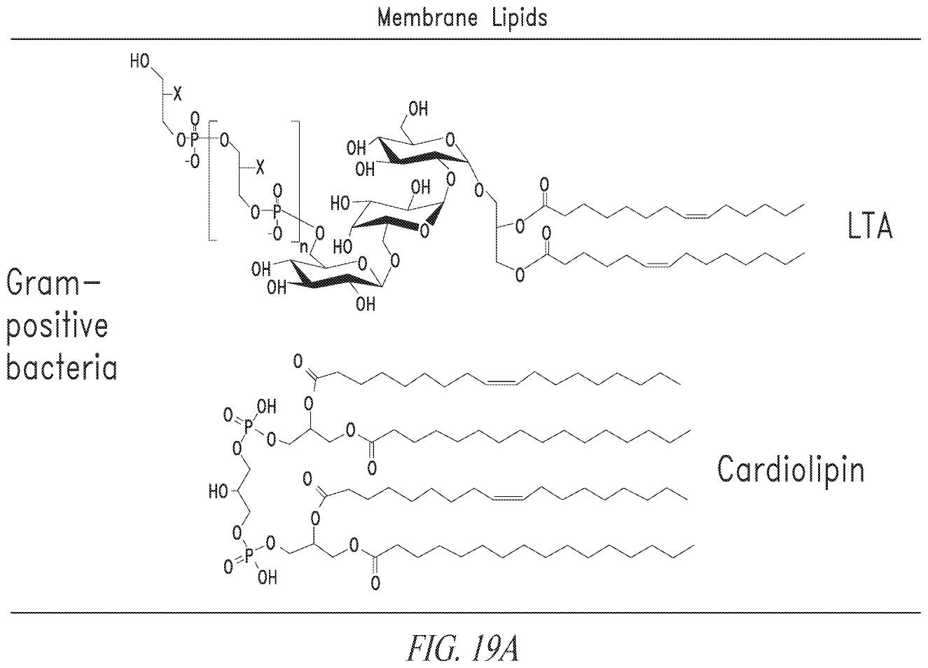

FIG. 18 depicts two detail views of a structured spot. The views of the structured spot are labeled “Top View” and “Bottom View.” In at least one embodiment, at least one structured spot is used with the surface visible in the top view facing upwards. In at least one embodiment, at least one structured spot is used with the surface visible in the bottom view facing upwards. The structured spot optionally contains a depression on at least one surface. In at least one embodiment, at least one depression is produced by etching away a base material upon which a structured spot has previously been self-assembled. In at least one embodiment, said depression is a convenience of the manufacturing process. In at least one embodiment, said depression contains or partially contains a volume of liquid placed in or on the depression, for example during a deposition, filtration, wash, or extraction process. In at least one embodiment, said depression on a surface of a structured spot prevents liquid that has passed through one or more pores from migrating on a surface of the structured spot and/or a plate containing it.

In at least one embodiment, at least one assembly contains a structured spot and at least one luer or similar fitting, and a liquid in a syringe, catheter, or similar is delivered by means of the at least one luer or similar fitting to the structured spot. In at least one embodiment, at least one measured volume of liquid in a syringe is delivered via an assembly to a structured spot. In at least one embodiment, a volume of liquid that is not precisely measured or a volume of liquid that is not measured at all is delivered by means of a luer or similar fitting to a structured spot.

### Embodiment 13: Clinical Sample

1. Any of the steps of Embodiments 1-12 are performed, using at least one sample that is a biological sample and/or a clinical sample.

In at least one embodiment, the biological sample is derived from a clinical specimen or sample. In at least one embodiment, at least one sample is obtained from a urine specimen, a blood sample, a sample incubated in a blood bottle, sputum, a sample obtained from sputum, feces, wound effluent, mucus, buccal swab, nasal swab, vaginal swab, nipple aspirate, sweat, saliva, semen or ejaculate, synovial fluid, bronchoalveolar lavage, tears, a urinary catheter sample, a culture plate, or another clinical or medical sample.

### Embodiment 14: Non-Clinical Sample

1. Any of the steps of Embodiments 1-12 are performed, using at least one sample that is a not a clinical sample.

In at least one embodiment, the non-clinical sample is an industrial sample, a environmental sample, an agricultural sample, a veterinary sample, a food sample, a forensic sample, a manufacturing process sample, a fermentation sample, a sterility sample, or any other other sample potentially containing a microbial organism.

### Embodiment 15: Culture Smear With Acid

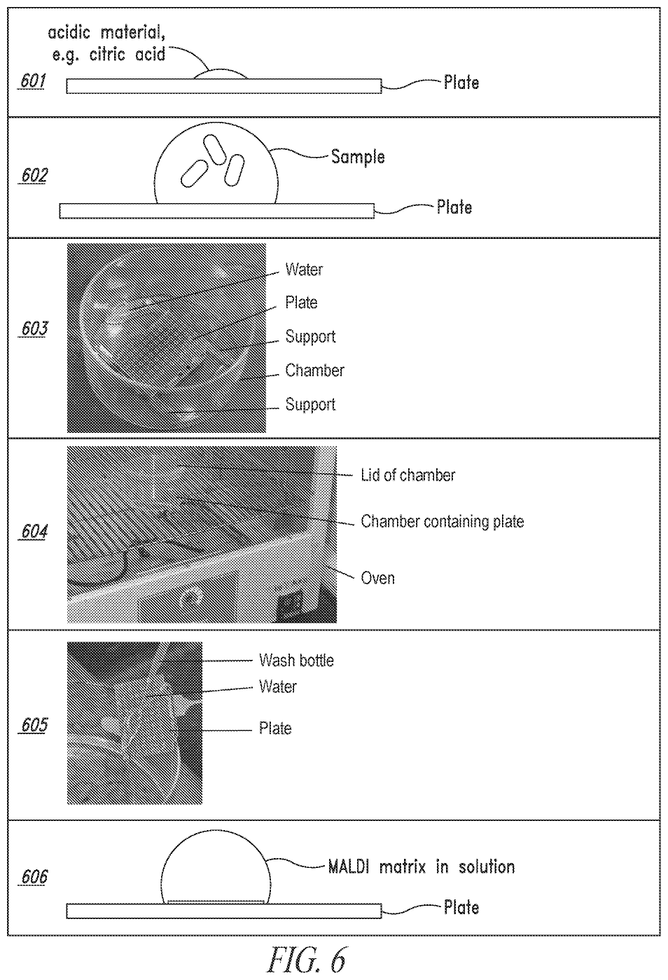

Perform the steps of Embodiment 1, as shown in of FIG. 6, as follows:

In step 601, obtain a plate, on which an acidic material such as, for example, dry citric acid has been deposited on one or more spots. Alternatively, obtain a plate and deposit an acidic material on one or more spots of the plate. In at least one embodiment, a liquid solution is or has been deposited and allowed to partly or entirely dry, leaving behind an acidic material.

In step 602, apply a sample comprising at least one smear sample of a colony from at least one culture plate onto at least one spot of the plate.

In step 603, place the plate in a chamber. In an embodiment, the chamber contains liquid. In an embodiment, the chamber contains water. In an embodiment, supports in the chamber hold the plate above the liquid, so that the plate is not immersed in the liquid. The chamber has a lid or is otherwise enclosed, so that the interior atmosphere is partially or entirely isolated from the atmosphere outside the chamber. In at least one embodiment, the chamber is not airtight. In at least one embodiment, the chamber is airtight. In at least on embodiment, the chamber is fitted with one or more instances of a valve, a pressure relief valve, a pressure interlock preventing sudden release of pressure, a port for introduction of liquid, gas, and/or vapor, a port for release of liquid, gas, and/or vapor, a pressure indicator, and/or a pressure sensor.

In step 604, heat the chamber in an oven, in an autoclave, on a hotplate, or any other heat source. In an embodiment, the chamber is heated to 110° C. for 30 minutes or 121° C. for 30 minutes.

In step 605, remove the plate from the chamber. Optionally allow the plate to cool. Wash the plate with a liquid. In an embodiment, the plate is washed with water squirted from a wash bottle. In at least one embodiment, the force of water striking the plate enhances the cleaning effect of washing.

In step 606, optionally apply a MALDI matrix in a solution or suspension to at least one spot on the plate. Allow to dry. In an embodiment, the matrix is norharmane. In an embodiment, the matrix is dissolved in a mixture containing chloroform and methanol.

### Embodiment 16: Culture Smear With Buffer

Perform the steps of Embodiment 15, except in step 601 a buffer solution is used in place of acid. In at least one embodiment, the steps of embodiment 16 are performed, except that in step 601 in place of acid apply about 1 μL of a solution containing citric acid at about 0.3M concentration and sodium citrate at about 0.3M concentration.

### Embodiment 17: Culture Smear with no Acid or Buffer

Perform the steps of Embodiment 15, except in step 601 no acid or buffer has been or is deposited.

### Embodiment 18: Escherichia coli and Maldi

The steps of Embodiment 15 were performed. In step 602, the colony comprised a colony of E. coli.

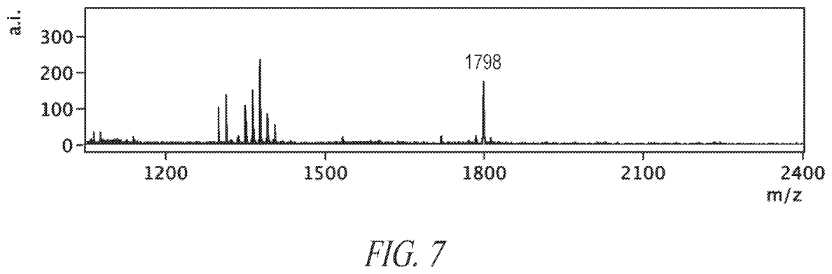

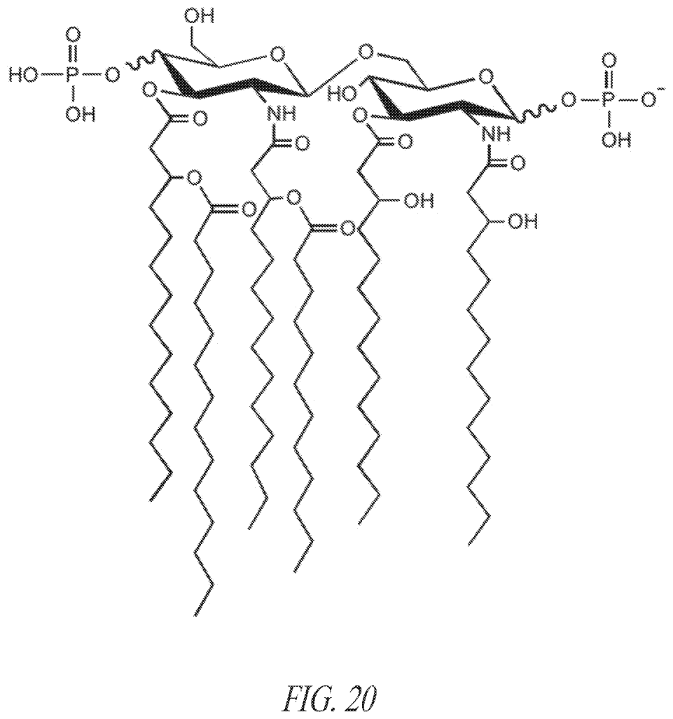

The plate was placed in a Bruker autoflex MALDI mass spectrometer. A spectrum from 1000 m/z to 2400 m/z was collected in negative ion mode, shown in FIG. 7. Said spectrum has ion peaks that are characteristic of lipids including lipid A from E. coli. For example, the peak at m/z 1798 is characteristic of E. coli and is attributed in the literature to a specific lipid A molecule of E. coli, shown in FIG. 20.

### Embodiment 19: Bacillus subtilis in Autoclave

1. The steps of Embodiment 15 were performed with modifications noted below. In step 602, the colony comprised a colony of B. subtilis. Different from Embodiment 15, in step 603, the plate was placed in glass petri dish. The glass petri dish containing the plate was placed in a sterilization autoclave and the top of the glass petri dish was propped up with a small metal block so that the atmosphere inside the glass petri dish was in communication with the atmosphere of the autoclave outside the glass petri dish. A standard gravity steam sterilization cycle was run on the autoclave, said cycle taking about 40 minutes to complete, and said cycle including a heat step of about 30 minutes at 121° C. No drying step was performed after the heat step. In an embodiment, a drying step is not used with an autoclave cycle, which can offer at least one of the advantages of saving time, reducing the risk of condensation on the plate, and improving signal quality. In at least one embodiment, a drying step is performed after a heat step, using an autoclave.

2. The plate was placed in a Bruker autoflex MALDI mass spectrometer. A spectrum from 800 m/z to 2400 m/z was collected in negative ion mode Said spectrum is shown in FIG. 8A, and contains characteristic ion peaks of lipids from B. subtilis.

Some of the aforementioned characteristic ions in FIG. 8A can be identified by comparison to FIG. 8C, which was produced by MALDI analysis of lipids extracted from B. subtilis using a Caroff-type extraction. Briefly, a sample of B. subtilis was lyophilized overnight, then incubated at approximately 100° C. in a solution containing isobutyric acid, the resulting extracted solution separated by centrifugation and washing. This Caroff-type extract was then analyzed on a Bruker microflex with an m/z range from 800 to 2400 Da, producing the spectrum in FIG. 8C.

### Embodiment 20: Bacillus subtilis l in Oven

The steps of Embodiment 19 were performed, except that in step 603, the container containing a plate was the chamber from Embodiment 15, and in step 604 the container containing the plate was heated in an oven at 110° C. for 30 minutes. The resulting spectrum is shown in FIG. 8B, which has characteristic features of lipids from B. subtilis. Some of the aforementioned characteristic ions in FIG. 8B can be identified by comparison to FIGS. 8A and 8C.

In combination, FIGS. 8A, 8B, and 8C show that embodiments of the present disclosure can produce many or all of the same characteristic B. subtilis ions in an oven and in an autoclave, and that these are also some or all of the the same characteristic ions obtained from B. subtilis via one or more similar methods using a Caroff-type extraction.

### Embodiment 21: E. coli by Three Methods

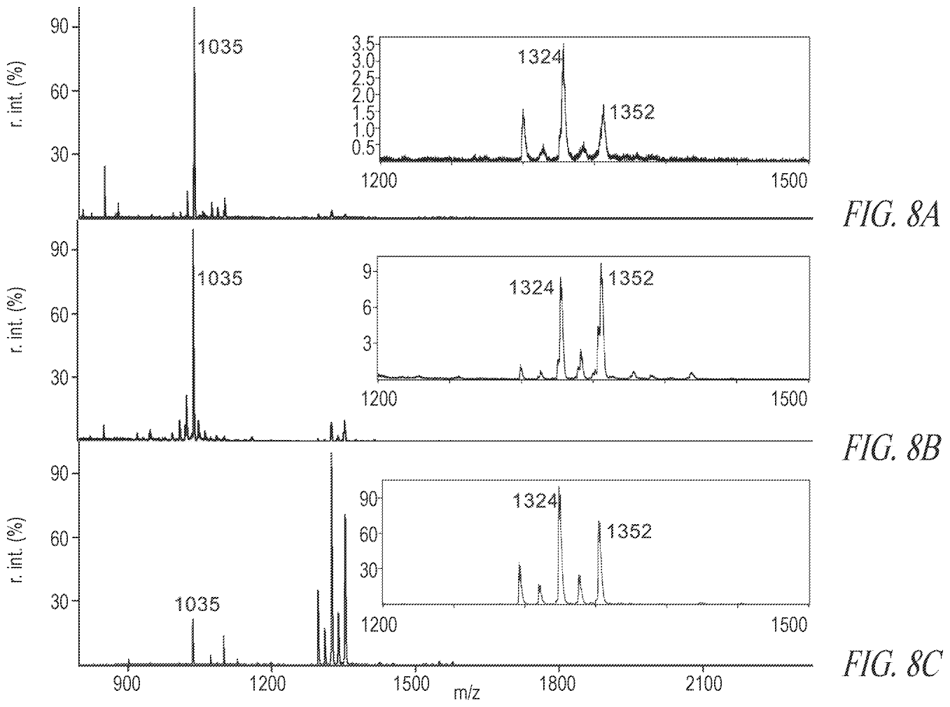

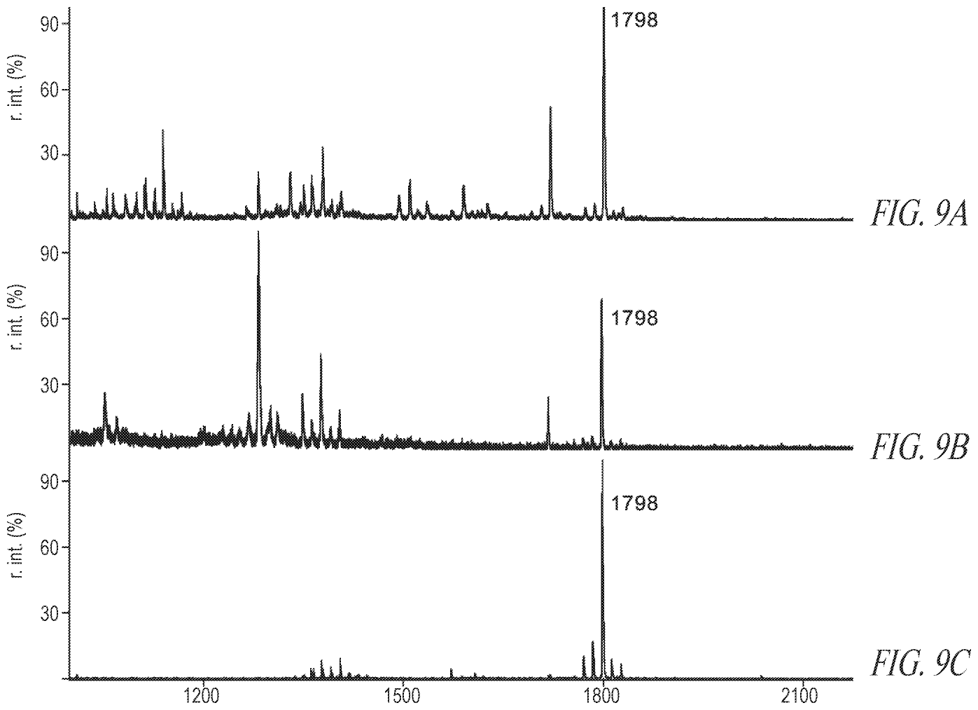

In FIG. 9, subfigures A, B, and C were produced by same methods used to produce the respective subfigures A, B, and C in FIG. 8, except that in FIG. 9 the colony comprised a colony of E. coli, and in FIG. 9 spectra were collected from 1000 to 2400 m/z. The spectra had essentially no ions of interest between 2200 and 2400 m/z, so the portions of spectra above 2200 m/z are not shown.

In combination, FIGS. 9A, 9B, and 9C show that embodiments of the present disclosure can produce many or all of the same characteristic E. coli ions in an oven and in an autoclave, and that these are also some or all of the the same characteristic ions obtained via one or more similar methods using a Caroff-type extraction.

### Embodiment 22: Lysozyme

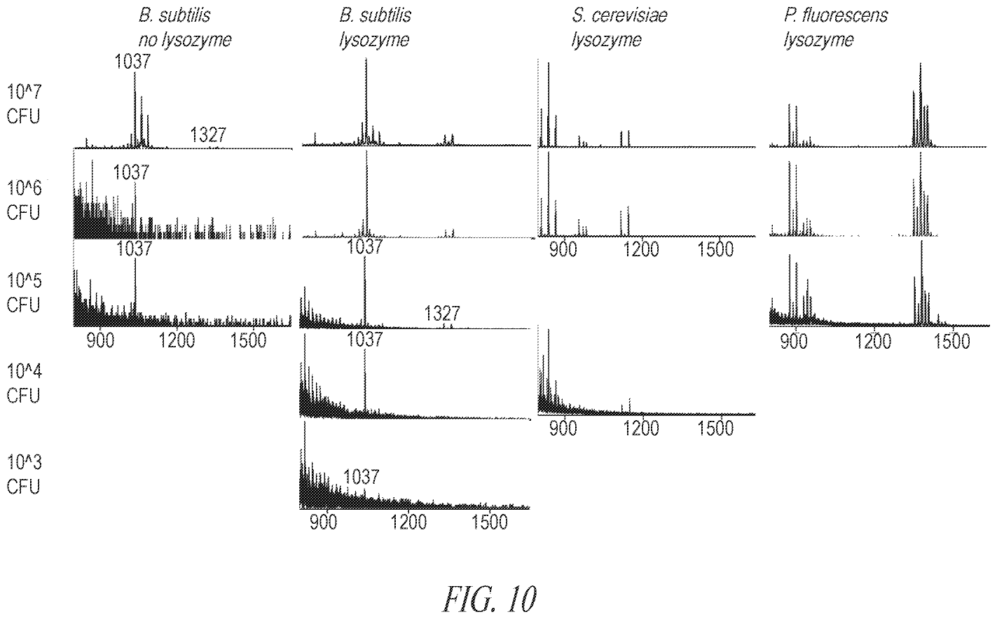

The methods used in Embodiments 15 through 21 were performed, except that multiple samples were processed on multiple spots producing multiple spectra as follows: (i) 1 μL of 2 mg/mL lysozyme was applied to some of the plate spots and allowed to dry; (ii) a liquid sample of each microbe mentioned in the column headings at the top of FIG. 10 was prepared, concentration in CFU/mL (colony forming units per mL) for each such said liquid sample was estimated by optical density, then for each such said liquid sample a log dilution series from 10¹⁰ to 10⁸, 10⁷, or 10⁶ was prepared; (iii) no acid or buffer was used, as in embodiment 18; (iv) in step 602, 1 μL of liquid from each dilution of each microbe was dispensed by pipette, delivering an estimated absolute quantity of between 10 ³ and 10 ⁷ CFU; (v) spectra were collected from 800 to 2400 m/z, but for clarity only the range 800 to about 1600 m/z is shown in FIG. 10.

Resulting spectra are shown in FIG. 10. For each spectrum, the species of the colony is as noted in the heading above the column in which the spectrum appears, and the estimated absolute number of CFU is as noted in the row heading to the left of the row in which the spectrum appears. All spectra were collected from spots that had lysozyme pre-dispensed on them as described above in (i), except that the leftmost column did not have any lysozyme.

FIG. 10 shows that one or more embodiments of the present disclosure result in a LOD as low as 10³ CFU, and produces most or all of the same characteristic ions across a wide concentration range.

Lysozyme was not shown to worsen LOD using the described method for the species shown in FIG. 10. Moreover, lysozyme appeared to improve LOD for B. subtilis by about 10×. Lysozyme partly breaks down the membranes of most Gram-positive bacteria, so in one or more embodiments of the present disclosure lysozyme may improve LOD for some or all Gram-positive bacteria. Lysozyme also affects Gram-negative and fungal membranes, so in one or more embodiments lysozyme improves LOD for organisms other than Gram-positive bacteria.

Also, as shown in FIG. 10, in at least one embodiment of the disclosure, extraction and analysis of membrane lipids from fungal organism are extracted and/or analyzed. For example without limitation, in at least one embodiment, the fungal organism is S. cerevisiae.

Herein, unless specified otherwise or clear from context, the terms “fungal” and “fungus” are in reference to a fungus, a yeast, a mold, and/or a member of the kingdom fungi.

In at least one embodiment, lysozyme is used in combination with a buffer, or in combination with an acidic material. In at least one embodiment, at least one sample is exposed to lysozyme before said buffer or acidic material is added. In at least one embodiment, more than one acidic and/or buffered condition is used.

### Embodiment 23: Classifying BCG Species

The steps of Embodiment 6 were performed. Briefly, the classification method used a machine learning model comprised of ensembles of support vector machines applied to features selected from mass spectra transformed via autocorrelation. The training data comprised mass spectra from every species of the Bacillus cereus Group (BCG), extracted with a Caroff-type extraction.

The test spectra that were classified comprised samples prepared using the steps of Embodiment 21, except that samples of every species in BCG were used.

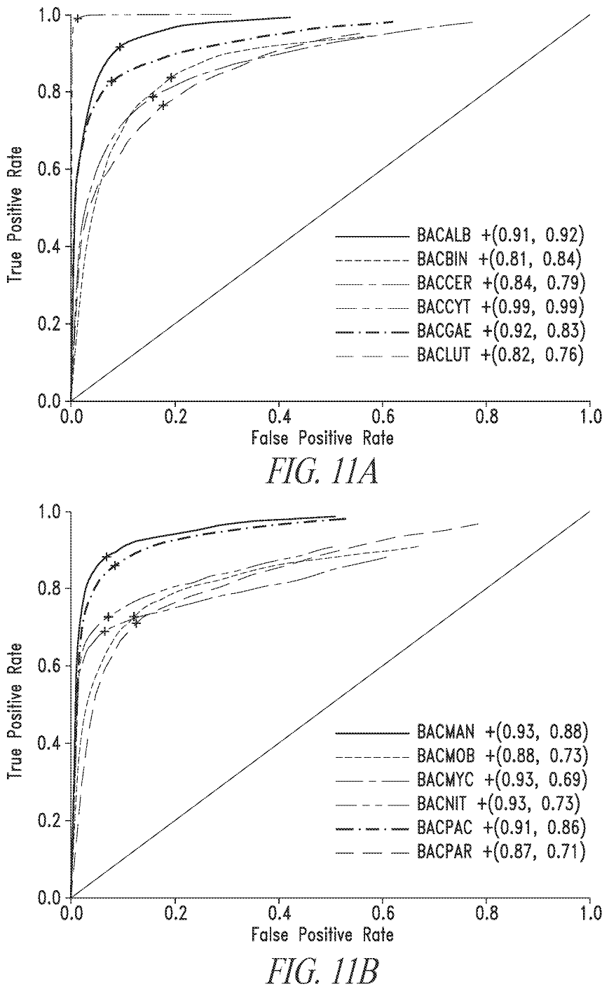

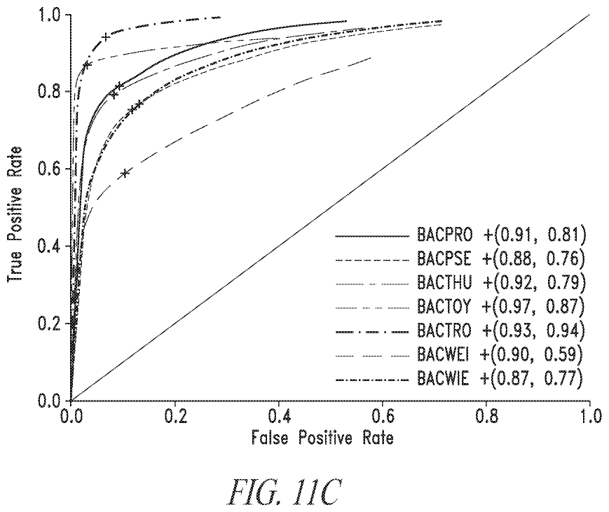

FIGS. 11A through 11C show ROC curves for samples of each BCG species. In the legend, species are noted by a 6-letter code, as follows: BACALB: Bacillus albus, BACBIN: Bacillus bingmayongensis, BACCER: Bacillus cereus, BACCYT: Bacillus cytotoxicus, BACGAE: Bacillus gaemokensis, BACLUT: Bacillus luti, BACMAN: Bacillus manliponensis, BACMOB: Bacillus mobilis, BACMYC: Bacillus mycoides, BACNIT: Bacillus nitratireducens, BACPAC: Bacillus pacificus, BACPAR: Bacillus paranthracis, BACPRO: Bacillus proteolyticus, BACPSE: Bacillus pseudomycoides, BACTHU: Bacillus thuringiensis, BACTOY: Bacillus toyonensis, BACTRO: Bacillus tropicus, BACWEI: Bacillus weihenstephanensis, BACWIE: Bacillus wiedmannii.

FIGS. 11A through 11C show that, in an embodiment of the present disclosure, spectra from every species except Bacillus anthracis in the closely-related BCG were identified and classified using machine learning models.

### Embodiment 24: Raman Spectroscopy

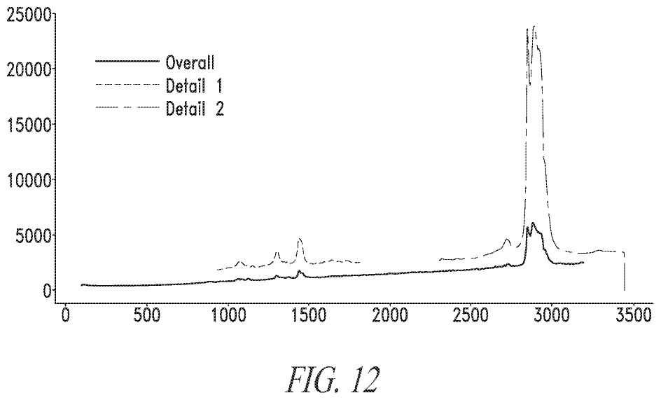

Lipids including lipid A were extracted from a sample of E. coli, using a Tao-type extraction, which is a rapid version of a Caroff-type extraction. In particular, a Tao-type extraction does not require lyophilization. A solution containing said extracted lipids was placed on a MALDI plate. As shown in FIG. 12, Raman spectra were collected from the plate using a 514 nm laser. In FIG. 12, the horizontal axis is wavenumber (1/cm) and the vertical axis is arbitrary intensity. The solid line curve shows an overall spectrum. The dotted detail 1 and detail 2 curves show details of specific wavenumber regions.

FIG. 12 shows that lipid A and/or other lipids addressed by the present disclosure produce discernable spectral peaks via Raman spectroscopy.

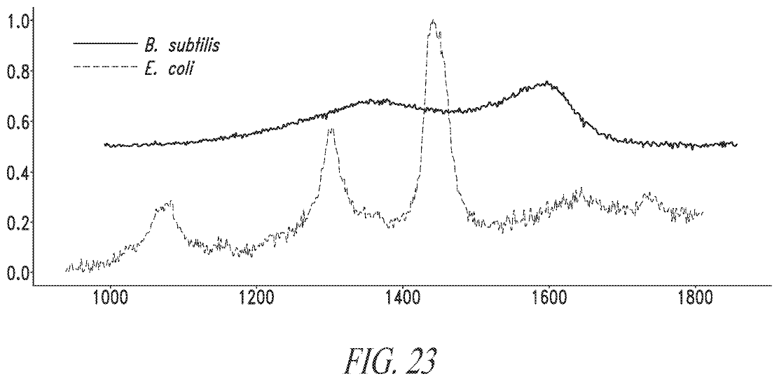

In FIG. 23 is shown two Raman spectra. The method of Embodiment 1 was used on a spot of a stainless steel plate to extract lipids produced by Bacillus subtilis. Raman spectra were collected from the spot, using a 514 nm laser. In FIG. 23, the horizontal axis is wavenumber (1/cm) and the vertical axis is arbitrary intensity. The two differently patterned curves are the spectrums for B. subtilis and for E. coli extracted using a Tao-type extraction, also shown in FIG. 12. The two curves in FIG. 23 show substantially different spectral peaks. One skilled in the art will appreciate that comparison of the B. subtilis and E. coli curves in FIG. 23 shows that the Raman peaks produced for B. subtilis are substantially different than Raman peaks produced for E. coli. One skilled in the art will further appreciate that differences in spectral peaks such as can be observed in FIG. 23 are consistent with the degree and type of spectral differences necessary to perform identification of microbial species present or not present in a sample, and/or identification of an organism at a taxonomic level other than species, such as without limitation distinguishing the presence or absence of one or both of at least one Gram-positive bacterium and at least one Gram-negative bacterium.

In at least one embodiment, at least one sample of lipids extracted by at least one method described in the embodiments or elsewhere herein is analyzed via Raman spectroscopy. In at least one embodiment, at least one sample of lipids extracted by at least one method other than one described in the embodiments or elsewhere herein is analyzed via Raman spectroscopy. In at least one embodiment, at least one sample of lipids extracted by Bligh-Dyer extraction, Caroff-type extraction, and/or Tao-type extraction is analyzed via Raman spectroscopy.

In at least one embodiment, one or more samples as described above for this embodiment are analyzed via Raman spectroscopy for any or all of the purposes described under the heading “Purposes For Extraction.”

### Embodiment 25: Enclosure

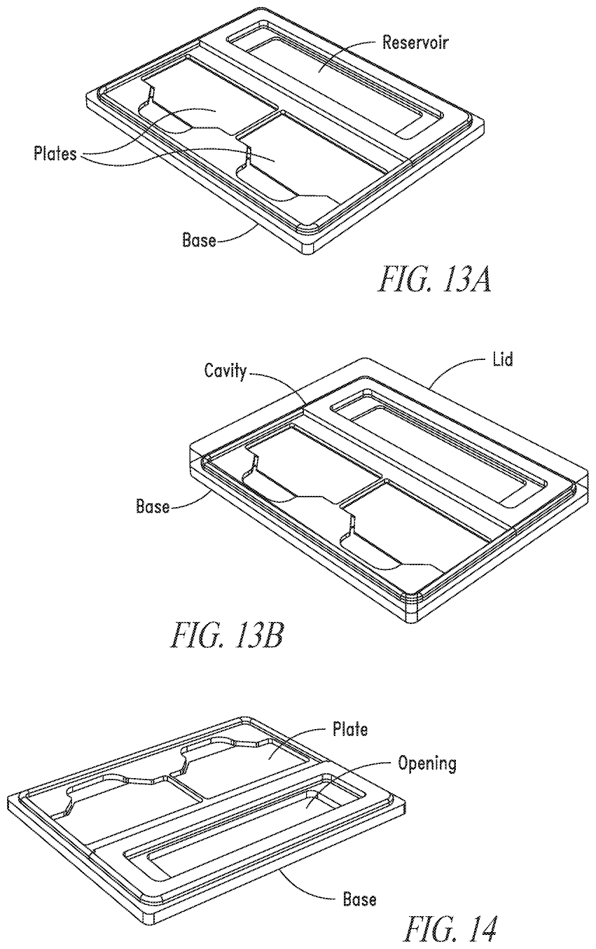

FIG. 13a depicts two plates placed in a holder base. In at least one embodiment, a holder base is made of aluminum, another metal, ultem, another polymer, or another material. In at least one embodiment, the base is made of a material that can repeatedly withstand temperatures higher than 100° C. One skilled in the art will appreciate that in at least one embodiment, a holder base holds 1, 2 or more than 2 plates. In at least one embodiment, a holder base contains a reservoir. In at least one embodiment, a reservoir in an enclosure contains a quantity of water. In at least one embodiment, said water is deionized (DI) water or is distilled water. In at least one embodiment, the quantity of said water is about 1 cc.

FIG. 13b depicts a base with two plates. A lid has been placed over the base, the lid and base together forming an enclosure. In at least one embodiment, a base is used that is a base as described above in reference to FIG. 13a. In at least one embodiment, a lid is used that is made of the same material as the base. In at least one embodiment, a lid is used that is made of a different material than the base. In at least one embodiment, a lid and base are constructed such that there is a cavity between the reservoir area and the area holding plates, said cavity allowing atmosphere to be exchanged between the reservoir and one or more plates. One skilled in the art will appreciate that in at least one embodiment, a cavity between a reservoir area and a plate area is constructed entirely within a single piece, such as a base or a lid.

In at least one embodiment, an enclosure such as shown in FIG. 13b is heated by being placed in an oven or autoclave, by being placed on a heat block or hotplate, by being subjected to electromagnetic radiation such as microwaves, radio waves, or infrared energy, or by other means accomplishing heating. In at least one embodiment, at least one enclosure has as one or more of its parts a heating element or other means of heating, so that the enclosure can be operated to heat itself, with or without applying heat from another device.

In at least one embodiment, an enclosure is used that is airtight. In at least one embodiment, an airtight enclosure is used that contains one or more instances of a valve, a pressure relief valve, a pressure interlock preventing sudden release of pressure, a port for introduction of liquid, gas, and/or vapor, a port for release of liquid, gas, and/or vapor, a pressure indicator, and/or a pressure sensor. In at least one embodiment, the aforementioned airtight enclosure is used for controlled pressurization and/or depressurization of said airtight enclosure.

In at least one embodiment, an enclosure is used that is not airtight. In an embodiment, the enclosure is not airtight so that as it is heated, water vapor partially or entirely displaces air inside the enclosure, but the interior of the enclosure remains at about the same air pressure as the environment around the enclosure.

### Embodiment 26: Vented Enclosure

FIG. 14 depicts an alternative base similar to the base shown in FIG. 13a, except that there is an opening in the said alternative base, said opening facilitating the exchange of atmosphere between the inside of the enclosure and outside of the enclosure. In at least one embodiment, a base such as shown in FIG. 14 is combined with a lid such as shown in FIG. 13b to form an enclosure. In at least one embodiment, an enclosure is used that is heated by any of the methods of Embodiment 26. In an embodiment, said enclosure is heated in an autoclave, or is situated in a similar heated humid environment. In an embodiment, the opening faces downwards to reduce the possibility of water dripping into the enclosure. In at least one embodiment, an enclosure is used that contains an opening that is not in a base part and/or does not open downwards.

In at least one embodiment, an enclosure includes an opening that is not part of a base part. In at least one embodiment an enclosure includes an opening that does not open downwards. In at least one embodiment, an enclosure includes both a reservoir and an opening. In at least one embodiment, an enclosure includes neither a reservoir nor an opening.

In at least one embodiment, an enclosure as described in embodiments 26 and/or 27, and/or as shown in FIG. 13b, and/or containing a base as shown in FIG. 13a and/or FIG. 14, and/or an enclosure having the equivalent function of same, is used to perform extraction of lipids and/or other substances in any of the ways described in the embodiments or otherwise described herein. One skilled in the art will appreciate that in at least one embodiment, various configurations of parts enclosing one or more plates and optionally containing a reservoir comprise an enclosure performing substantially the same function of enclosing as shown in FIGS. 13a, 13b, and 14.

### Embodiment 27: Pre-Dispensed Plate

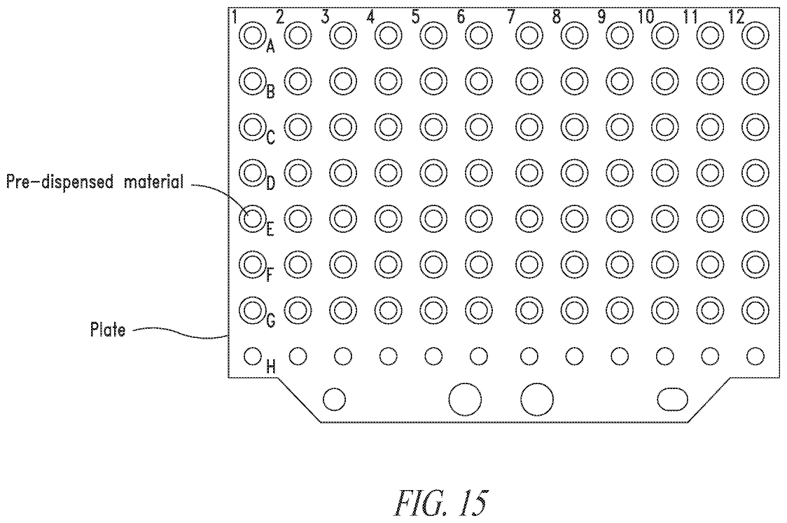

FIG. 15 depicts a plate with material pre-dispensed on it. In at least one embodiment, a material is pre-dispensed onto one or more spots of a plate. In at least one embodiment, a material is pre-dispensed, said material comprising any or all of an acidic solid, a buffer, an enzyme, an emulsifier, a detergent, and a hydrogel. In at least one embodiment, a plate with pre-dispensed material is distributed. In at least one embodiment, a plate with or without pre-dispensed material is distributed, and then a material is pre-dispensed onto at least one spot of a plate prior to or after applying sample to said spot. In at least one embodiment, at least one material is pre-dispensed onto a structured spot, then the spot is optionally attached to a plate. In at least one embodiment, one or more structured spots with or without pre-dispensed material are distributed, and then a material is pre-dispensed onto at least one structured spot.

As used herein “pre-dispensed” means applied or dispensed before performing some other operation, such as a method or a step of a method described herein.

### Embodiment 28: More than One Pre-Dispensed Material

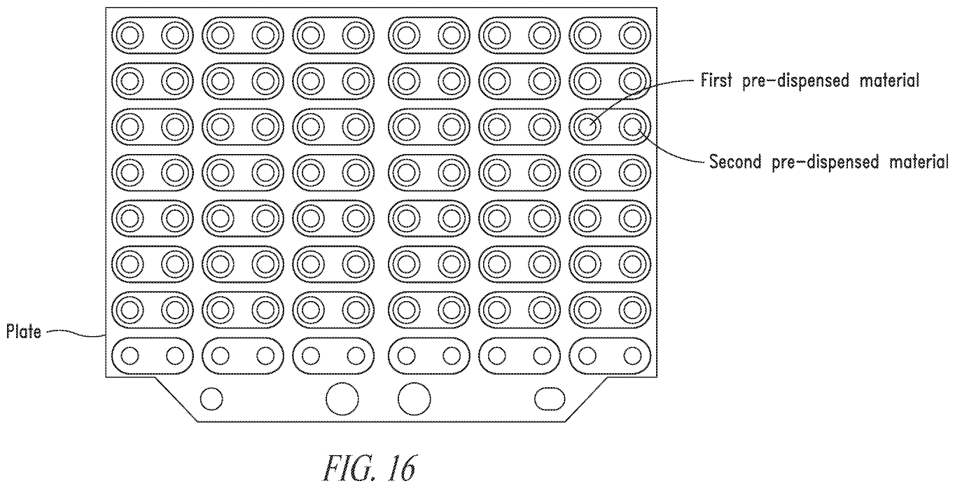

FIG. 16 depicts a plate with a first material and a second material pre-dispensed on it. In at least one embodiment, for at least one group of spots, at least one of the aforementioned materials is dispensed on at least one spot in a group of at least two spots. One skilled in the art will appreciate that in at least one embodiment, more than one material is pre-dispensed on one or more single spots, and/or one material is pre-dispensed on more than one spot in at least one group of spots. In at least one embodiment, at least one group of spots comprises 3 or more spots. In at least one embodiment, three or more different materials are pre-dispensed on one or more spots, said spots optionally comprising or belonging to at least one group of spots. In at least one embodiment, at least one material is pre-dispensed on at least one structured spot, then the at least one structured spot is optionally attached to a plate. In at least one embodiment, for at least one group of spots, a material dispensed on at least one first spot in the group of spots has a different composition and/or volume than a second material dispensed on at least one second spot in the group of spots.

In at least one embodiment, for at least one group of spots, a sample is applied to at least two spots in a group of spots.

In at least one embodiment, a material that is dispensed on at least one spot preferentially extracts lipids of a particular class, preferentially extracts lipids from one kind of microbe, and/or preferentially binds or retains one or more lipids, lipid membranes, or parts of lipid membranes, said spot optionally belonging to at least one group of spots.

For example without limitation, for at least one group of spots, in an embodiment, at least one spot in the at least one group of spots is pre-dispensed with a buffer comprising a mixture of about 1:1 molar concentration of citric acid and trisodium citrate, while at least one other spot in the group of spots does not have any material pre-dispensed on it. In at least one embodiment, for Gram-negative bacteria, the said spot with buffer will have a relatively low pH, facilitating cleavage of the Kdo bond in lipopolysaccharide (LPS) and/or facilitating extraction of lipid A. In at least one embodiment for at least one microbe that is not a Gram-negative bacterium, the said first and second spots will facilitate extraction of different membrane lipids; methods of the present disclosure are then performed using either the first spot spot or the second spot, or using both the first spot and the second spot, or using additional spots in addition to the first spot and/or the second spot.

For example without limitation, in an embodiment, for at least one group of spots, at least one spot in the group of spots is pre-dispensed with lysozyme, while at least one spot in the group of spots is not pre-dispensed with lysozyme. Lysozyme disrupts the membranes of microbes by multiple actions. For example, lysozyme disrupts the membranes of Gram-positive bacteria by catalyzing the hydrolysis of peptidoglycan. In at least one embodiment, lysozyme modifies the extraction of lipids from at least one microbe. In an embodiment, lysozyme modifies the extraction of lipids from at least one Gram-positive bacterium, improving the LOD for said at least one Gram-positive bacterium. However, at least one chemical activity of lysozyme depends on the pH or alternatively on the acidity of the mixture comprising it. At typical conditions, lysozyme derived from chicken egg is most effective in catalyzing the hydrolysis of peptidoglycan in the membrane of at least one Gram-positive bacterium at a pH of roughly 6.0; typically, said lysozyme is progressively less effective at pH values less than about 6.0, having substantially reduced effectiveness at a pH of about 5.0. Conversely, at typical conditions, optimal cleavage of the Kdo bond in LPS is at a pH of about 4.0 or less than about 4.0. Consequently, in at least one embodiment, at least two spots with different pH values or acidity will each preferentially extract different lipids of interest from microbes. Further, in at least one embodiment, on at least one spot, lysozyme at a pH of between about 5.0 and about 7.0 preferentially extracts at least one lipid from at least one Gram-positive bacterium, whereas on at least one other spot, a pH of about 4.0 or less preferentially extracts lipid A from at least one Gram-negative bacterium.

For example without limitation, in at least one embodiment, for at least one group of spots, at least one spot in the at least one group of spots is pre-spotted with different material than at least one other spot. In at least one embodiment, for at least one group of spots, at least one spot in the at least one group of spots is not pre-spotted with any material and at least one other spot in said group of spots is pre-spotted with material. In said example, in at least one embodiment, compared to a single spot, said group of spots improves at least one of the limit of detection, signal quality, or classification accuracy of at least one of two or more microbial species when said at least one microbial species is present in a sample. In said example, in at least one embodiment, compared to a single spot, said group of spots improves at least one of the limit of detection, signal quality, or classification accuracy of at least one of at least two microbial species when both said microbial species are present in a single sample. As a further example without limitation, for at least one group of spots, the group of spots contains two spots: a first spot is pre-dispensed with a citric acid buffer and a second spot is pre-dispensed with lysozyme. The aforementioned first spot provides a more desirable extraction of lipids from E. coli, which may be observed as one or more of improved spectrum quality of at least one MALDI spectrum obtained from E. coli, improved signal to noise ratio of said spectrum, greater signal intensity of higher mass ions in said spectrum, better signal intensity of an ion at m/z of about 1798 Da corresponding to a lipid A of E. coli in said spectrum, and improved sensitivity and/or specificity in classifying one or more E. coli samples based on said spectrum. The aforementioned second spot provides a more desirable extraction of at least one Gram-positive bacterium, which may be observed as one or more of improved spectrum quality of at least one MALDI spectrum obtained from said at least one Gram-positive bacterium, improved signal to noise ratio of said MALDI spectrum, greater signal intensity of at least one ions in said spectrum, and improved sensitivity and/or specificity in classifying one or more samples of said at least one Gram-positive bacterium based on said spectrum.

As a further example without limitation, a group of two or more spots with at least one spot in said group of two or more spots having a different composition of pre-dispensed material as discussed above, and the combined spectra obtained from two or more spots in said group of at least two spots and/or the combination of features or other data obtained from spectra of said at least two spots comprises more desirable data than the same obtained from either spot separately.

As one skilled in the art will appreciate, in at least one embodiment, markings on the plate indicate to an operator how at least two spots are grouped on a plate, such markings facilitating dispensing of samples by hand. In at least one embodiment, samples are dispensed using a fixture that facilitates dispensing of samples. In at least one embodiment, samples are dispensed onto a plate by automated equipment with or without a capability for optical alignment.

### Embodiment 29: Gasket

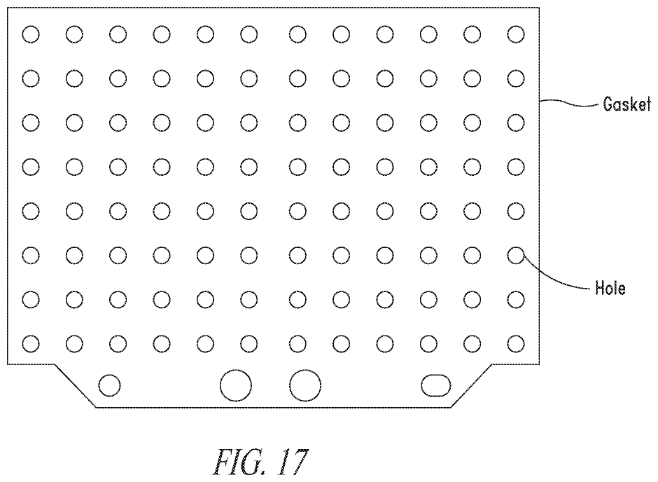

FIG. 17 depicts a top view of a gasket, said gasket having at least one hole that in at least one embodiment is aligned with at least one spot on a plate. In an embodiment, a gasket is made of silicone or a similar material such that, when said gasket is placed on a plate, liquid applied to at least one hole in the gasket is confined to the area around at least one hole on the plate, corresponding to the area around one or more spots on the plate. In at least one embodiment, a gasket is aligned to a plate via the edges of the gasket and plate; or via at least one hole, slot, marking, or edge of the plate, and at least one hole, slot, marking, or edge of the gasket. In at least one embodiment, a gasket is placed on a plate while said plate is washed. In at least one embodiment, a gasket is placed on a plate and at least one sample is dispensed onto at least one spot of said plate. In at least one embodiment, a gasket is placed on a plate while one or more materials are pre-dispensed onto at least one spot of the plate.

### Embodiment 30: Membrane Lipids

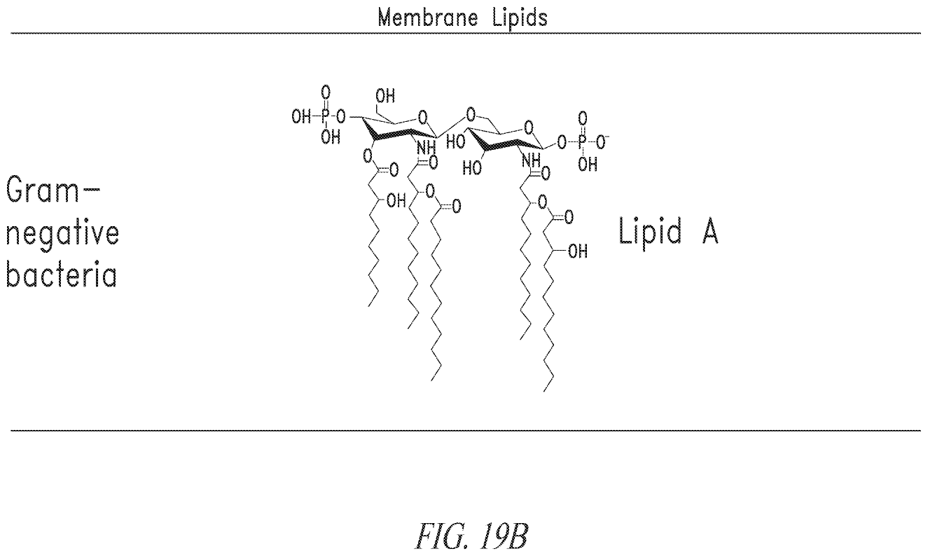

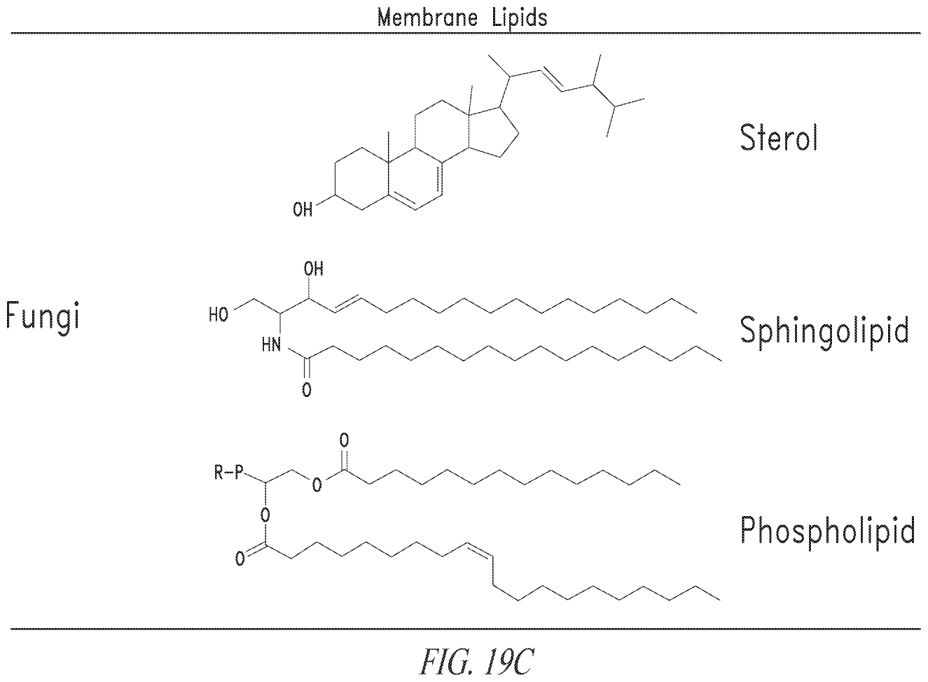

FIGS. 19A through 19C depict example lipid types for three classes of microbes: Gram-positive bacteria, Gram-negative bacteria, and fungi. In at least one embodiment, for said classes of microbes, for at least one of said class of microbe, at least one type of said example lipid types is extracted. In at least one embodiment, for said classes of microbes, for at least one of said class of microbe, at least one lipid is extracted that is not shown in FIGS. 19A through 19C as an example lipid type for said class of microbe. For example without limitation, in at least one embodiment, phospholipids and/or sphingolipids are extracted from Gram-negative bacteria and/or Gram-positive bacteria, regardless that an example phospholipid and sphingolipid is each shown in FIG. 19C for fungi but not FIG. 19A for Gram-positive bacteria nor FIG. 19B for Gram-negative bacteria. As a further example without limitation, in at least one embodiment, cardiolipins are extracted from Gram-negative bacteria and/or fungi, regardless that an example cardiolipin is shown in FIG. 19A for Gram-positive bacteria but not in FIG. 19B for Gram-negative bacteria nor FIG. 19C for fungi.

### Embodiment 31: Lipid A

FIG. 20 depicts an example structure of lipid A. This structure is associated in the literature with lipid A from E. coli, observed at a nominal mass of about 1798 Da.

### Embodiment 32: Dispensing onto Structured Spot

FIG. 21 depicts a plate to which are attached one or more structured spots. A liquid dispenser such as a pipette dispenses liquid onto the spot. Some or all of the liquid passes through the pores in the structured spot. In at least one embodiment the aforementioned process accomplishes one or more of depositing at least one substance suspended and/or dissolved in the liquid onto the surface of the spot, dissolving and/or suspending at least one substance on the surface of the spot, then carrying said substance through the pores of the spot, and/or moving at least one substance on one surface of the spot to the other surface of the spot; wherein the aforementioned at least one substances can in each instance be the same at least one substance or a different at least one substance as the case for a different instance.

One skilled in the art will appreciate that, in at least one embodiment, the process of Embodiment 32 is performed on at least one spot that is not attached to a plate.

In at least one embodiment, the process of Embodiment 32 is used to filter microbes out of at least one solution such as without limitation a urine specimen.

In at least one embodiment, the process of Embodiment 32 is used to filter cells and/or objects larger than the size of some or all microbes out of a solution.

In at least one embodiment, the process of Embodiment 32 is used to concentrate microbes out of a solution with a volume greater than about 1 μL, about 1 μL, or less than about 1 μL.

In at least one embodiment, the process of Embodiment 32 is used to remove one or more salts from at least one sample.

In at least one embodiment, the process of Embodiment 32 is used to separate water-soluble materials from materials that are not water soluble or have low water solubility. In at least one embodiment, the process of Embodiment 32 is used to separate all or some lipids from at least one sample. In at least one embodiment, the process of Embodiment 32 is used to collect water soluble materials including proteins from at least one sample.

In at least one embodiment, the process of Embodiment 32 is used to first concentrate microbes out of a solution with a volume greater than about 1 μL. Microbial lipids are then extracted using the process of embodiment 1 or any other process described herein. The process of Embodiment 32 is then used to wash some or all predominantly water-soluble materials off or through a spot, such water-soluble materials including for example without limitation proteins.

In at least one embodiment, the process of Embodiment 32 is performed by dispensing a liquid in a downward direction onto the top surface of a spot. In at least one embodiment, the process of Embodiment 32 is performed by dispensing a liquid in a direction other than downward and/or dispensing a liquid on to a surface other than the top surface of a spot.

In at least one embodiment, the process of Embodiment 32 is performed by dispensing a vapor which partly or entirely condenses into a liquid, or by dispensing a liquid which partly or entirely evaporates into a vapor. In at least one embodiment, the process of Embodiment 32 is performed by dispensing a colloid, slurry, paste, powder, gel, foam, or other material.

In at least one embodiment, the process of Embodiment 32 is applied using more than one liquid, or more than one substance at least one of which is not a liquid.

In at least one embodiment, the process of Embodiment 32 is applied, except replacing at least one liquid, with a non-liquid, such as without limitation a solid, paste, powder, semisolid, gel, foam, or slurry.

In at least one embodiment, the method of Embodiment 32 is applied to a sample consecutively using two or more structured spots, and/or is subjected to a filtration or separation step using a filter or separator that is not a structured spot before and/or after the process of Embodiment 33.

One skilled in the art will appreciate that, in at least one embodiment, liquid dispensed on one surface of a structured spot is prevented in part or entirely from passing through pores to the opposite surface of the structured spot, by placing a gasket, plug or similar on said opposite surface, or by clogging some or all pores, for example without limitation with a glue-like material.

### Embodiment 33: Kits

In at least one embodiment, at least one kit is distributed, said kit consisting in part or whole of one or more instances of at least one of the following: a plate, a plate with material pre-dispensed on it using any method described herein or by any other method, a plate containing at least one structured spot, a plate to which at least one structured spot can be attached, a structured spot, a gasket as described herein or of another type, a buffer solution, an acidic solution, a solid to which a liquid can be added to form a buffer or acidic solution, a matrix solution, a solid to which a liquid can be added to form a matrix solution, an enclosure as described in Embodiments 26 and/or 17 and/or elsewhere herein, a structured spot assembly as shown in FIGS. 4 and/or 5 and/or described elsewhere herein, instructions, and a software program and/or license key and/or other rights or credentials to a software program or software service that with or without additional data classifies samples and/or spectra according to any of the embodiments and/or any of the methods described herein.

In at least one embodiment, a kit contains at least one object not mentioned in the preceding description of Embodiment 33.

A “structured spot assembly” means at least any of an assembly containing a structured spot, a structure as shown in FIGS. 4 and/or 5, a collection of two or more parts that when assembled constitute a structured spot assembly, or a collection of one or more parts that when assembled with one or more additional parts constitutes a structured spot assembly. For example without limitation, a collection of parts not including a structured spot that when assembled with a structured spot constitutes a structured spot assembly is a structured spot assembly.

### Embodiment 34: Wells

FIG. 22 depicts shown a plate containing wells. In at least one embodiment, a plate containing at least one well is used to practice at least one method described in Embodiments 1-33 or elsewhere herein. In at least one embodiment, a plate containing at least one well contains at least one spot. In at least one embodiment, a plate containing at least one well contains at least one structured spot. In at least one embodiment, a plate contains at least one well with a volume of less than about 40 μL, between about 40 μL and about 50 μL, between about 50 μL and about 75 μL, between about 75 μL and about 100 μL, between about 100 μL and 150 μL, between about 150 μL and 200 μL or more than about 200 μL.

In at least one embodiment, a liquid is added to the aforementioned at least one well, and lipids and/or other molecules extracted onto a spot are dissolved and/or suspended into the liquid.

In at least one embodiment, a plate with at least one well is used to practice the method of Embodiment 2, and liquid from the well is injected, nebulized, or otherwise delivered into a mass spectrometer. For example, without limitation, in at least one embodiment liquid from the aforementioned well is injected into an electrospray mass spectrometer.

In at least one embodiment, a plate with at least one well is used to practice the method of Embodiment 3, and liquid from the well is injected, nebulized, or otherwise delivered into a spectroscopic instrument. For example, without limitation, in at least one embodiment, liquid from the aforementioned well is injected into an infrared spectroscopy instrument.

In at least one embodiment, at least one well contains one or more structured spots containing one or more pores, such that liquid can pass through one or more pores, thereby filling and/or draining the at least one well. One skilled in the art will appreciate that a plate containing the aforementioned at least one well resembles a “filter bottom”-type microtiter plate.

In at least one embodiment, a plate containing at least one well containing one or more spots and/or structured spots is used to practice at least one of the methods of Embodiments 1-33 or otherwise described herein.

For example without limitation, in at least one embodiment, the method of Embodiment 2 is performed using the aforementioned plate containing at least one well. A quantity of liquid is introduced into at least one well, and analytes of interest become dissolved or suspended in the quantity of liquid. At least some of the quantity of liquid is removed from the well by means familiar to those skilled in the art, and part or all of the said at least some quantity of liquid is analyzed by Embodiment 2 or 3 or any other embodiment herein or any other method described herein.

### Embodiment 35: Spatial Information

Perform at least one of the following steps:

1. Perform the method of one or more of Embodiments 2, 3, 4, 5, or 6. However, in addition to spectral information, capture information about the physical location on the plate from which one or more spectra were obtained.

2. Perform the method of one or more of Embodiments 2, 3, 4, 5, or 6. Obtain spectra from multiple locations on a spot, and segregate spectra by location on the spot, by one or more of at least the methods of combing only spectra that were collected at the same or substantially the same location, clustering spectra by location, and clustering spectra by relative similarity.

3. Perform the method of one or more of Embodiments 1-6 or 8-12. In at least one embodiment, at least two affinity reagents are placed in two or more respective spots, or else two or more of the at least two affinity reagents are placed in a single spot and any remaining affinity reagents are placed in the same or different spots.

In at least one embodiment, for at least one lipid or other analyte of interest, a pattern of distribution is detected. In at least one embodiment, said pattern is caused by one or more of at least one interaction with an affinity reagent, at least one concentration gradient, at least one concentration gradient that causes analytes to form in a predictable spatial pattern in a spot, one or more lipid rafts and/or similar structures becoming attached to the spot, at least one variation in mutual solubility of analytes of interest and/or other chemicals driving self sorting, or another reason.

### Embodiment 36: Standards

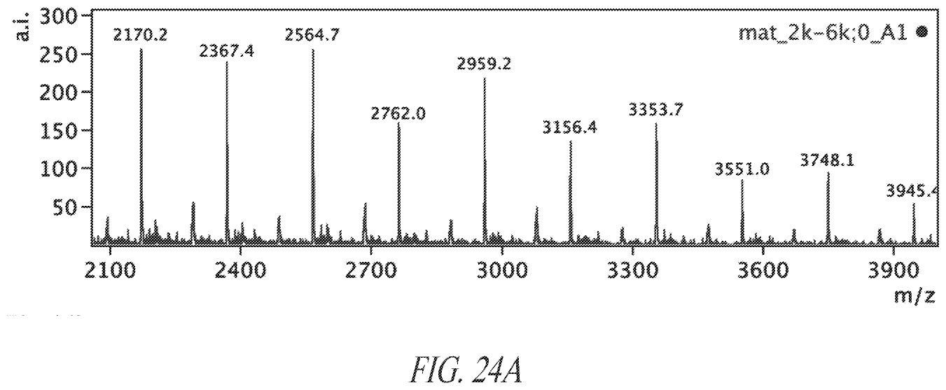

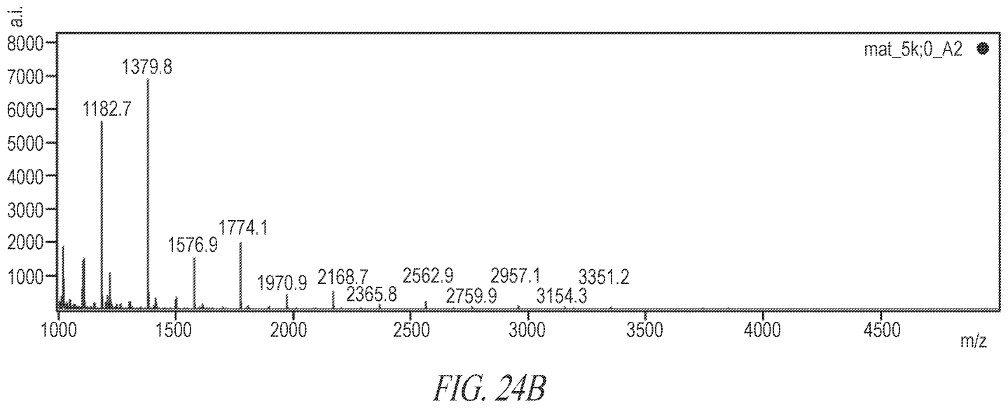

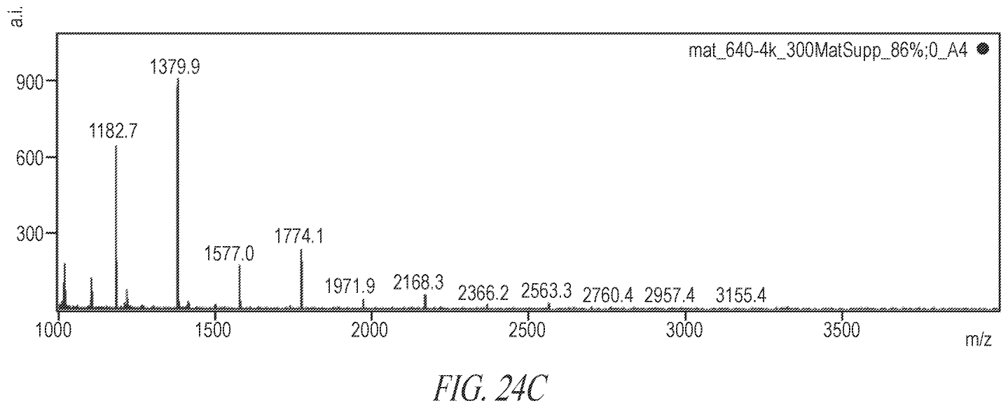

Gold-tin oxide nanoparticles (Au—SnO—NPs) were prepared and placed on spots on a MALDI plate. The MALDI plate was analyzed via MALDI mass spectrometry using a Bruker autoflex in negative ionization mode, producing the raw, uncalibrated spectra in FIGS. 24a-c. In FIGS. 24a-c, gold complex ion peaks are shown with a regular m/z spacing of about 198 Da, indicating that each successive ion peak differs from the preceding ion peak by the mass of a gold atom. This regular spacing facilitates quadratic or other nonlinear calibration of a mass spectrometer. One skilled in the art will appreciate that in at least one embodiment, a material other than gold-tin oxide is used as above as a MALDI matrix.

The aforementioned Au—SnO—NPs were made in approximately 2011, placed in a glass bottle with ordinary air and stored, then used as described herein in 2018, showing that nanoparticles such as Au—SnO—NPs can have long shelf lives.

In at least one embodiment, Au—SnO—NPs and/or other NPs are pre-dispensed on a plate or embedded in a plate, or Au—SnO—NPs and/or other NPs are applied to a plate but not pre-dispensed, and/or Au—SnO—NPs and/or other NPs comprise part of a matrix solution or composition, said matrix solution or composition applied by any method of matrix application described herein.

In one or more embodiments, Au—SnO—NPs and/or other NPs produce one or more calibration spectra and/or produce calibration ion peaks in a sample spectrum. One skilled in the art will appreciate that in at least one embodiment the abovementioned calibration spectra and/or calibration ion peaks are used to calibrate a mass spectrometer, to align or correct a spectrum, and/or to align or correct an integral transformed spectrum and/or one or more features, spectra, vectors, or other data as described herein. One skilled in the art will appreciate that in at least one embodiment, one or more internal and/or external standards are used to improve the accuracy and/or resolution of data, allowing one or more of improved accuracy of measurements of features, lower limit of detection, more accurate classification of samples, better detection and resolution of polymicrobial samples, faster processing of samples, and faster analysis of samples on an instrument and/or computationally. One skilled in the art will further appreciate that in at least one embodiment a material that is a standard and has a shelf life at room temperature of days, months, or years offers at least the advantages of easier handling of said standard, fabricating said standard into a plate and/or spot, including said standard in a matrix solution or other solution, or another method described herein.

### Embodiment 37: OMV AND MV

Perform the method of one or more of Embodiments 1-36 above. In at least one embodiment the methods are performed on a bacterial outer membrane vesicle (OMV), a bacterial membrane vesicle (MV), or any other membrane.

### Embodiment 38: Environmental Response

Perform the method of one or more of Embodiments 1-37 above. In at least one embodiment, a response of at least one cell, cell culture, tissue, microbial community, biofilm, or similar is determined and/or measured. In at least one embodiment, a response is determined and/or measured for the purpose of determining and/or measuring antibiotic resistance and/or susceptibility. In at least one embodiment a response is determined and/or measured for the purpose of determining and/or measuring one or more of an ADME-Tox (absorption/administration, distribution, metabolism, excretion, and toxicity) parameter or other measure, efficacy, release profile, toxicity, toxicology, and/or drug treatment effect for at least one substance.

This disclosure also includes the following embodiments:

Embodiment A. A method for extracting phospholipids and/or other analytes from a sample containing microbial organisms, the method comprising:

placing a quantity of liquid from the sample in contact with at least one surface, wherein the at least one surface is enclosed to facilitate control of evaporation of the quantity of liquid;

heating the quantity of liquid on the at least one surface;

optionally washing the at least one surface;

optionally allowing the at least one surface to at least partially dry; and

optionally applying a composition comprising at least one solvent and an optional substance having the property of acting as a MALDI matrix.

Embodiment B. A method for detecting the presence of at least one microbial organism in a sample, the method comprising:

placing a quantity of liquid from the sample in contact with at least one surface, wherein the at least one surface is enclosed to facilitate control of evaporation of the quantity of liquid;

heating the quantity of liquid on the at least one surface;

optionally washing the at least one surface;

optionally allowing the at least one surface to at least partially dry;

optionally applying a composition comprising at least one solvent and an optional substance having the property of acting as a MALDI matrix;

analyzing the contents of the at least one surface with at least one analytical instrument; and

determining or estimating the presence, quantity, taxon, or other property of the at least one microbial organism in the sample using at least one spectrum produced by the at least one analytical instrument.

Embodiment C. The method according to embodiment B, wherein the taxon or the other property of the at least one microbial organism is a bacterial species; a yeast and/or fungal species; a microbial species other than bacteria, a yeast, or a fungi; a microbial strain that is resistant, intermediately resistant, or susceptible to one or more antimicrobials, classes of antimicrobials, combinations of antimicrobials, and/or combinations of classes of antimicrobials; and wherein the other property is a microbial taxonomic classification above the level of species; a microbial taxonomic classification below the level of species, or a Gram stain classification.

Embodiment D. The method according to embodiment B, wherein the at least one analytical instrument comprises a mass spectrometer, optionally a MALDI mass spectrometer, a Raman spectrometer, or a spectroscopic instrument.

Embodiment E. The method according to any one of embodiments A-D, wherein the heating comprises heating the quantity of liquid in contact with the at least one surface to less than 95° C., about 95° C., about 100° C., about 110° C., about 121° C., about 125° C., about 130° C., about 135° C., or more than 135° C.

Embodiment F. The method according to any one of embodiments A-E, wherein the quantity of liquid is heated for less than 15 minutes, about 15 minutes, about 20 minutes, about 25 minutes, about 30 minutes, or longer than 30 minutes.

Embodiment G. The method according to any one of embodiments A-F, wherein the quantity of liquid has a volume of less than about 0.5 μL, about 0.5 μL, about 1.0 μL, about 1.5 μL, about 2 μL, about 3 μL, about 4 μL, about 6 μL, about 8 μL, about 10 μL, about 20 μL, or greater than 10 μL.

Embodiment H. The method according to any one of embodiments A-G, wherein the quantity of liquid contains microbial organisms at a concentration of greater than 109 Colony Forming Units (CFU)/mL, about 109 CFU/mL, about 108 CFU/mL, about 107 CFU/mL, about 106 CFU/mL, about 105 CFU/mL, about 104 CFU/mL, about 103 CFU/mL, about 102 CFU/mL, about 10 CFU/mL, about 1 CFU/mL, or less than 1 CFU/mL, or wherein the quantity of liquid contains no microbial organisms.

Embodiment I. The method according to any one of embodiments A-H, wherein the heating comprises heating the quantity of liquid by any one of the following: heating the at least one surface, heating the atmosphere surrounding the quantity of liquid , directly heating the quantity of liquid, and/or any combination thereof.

Embodiment J. The method according to any one of embodiments A-I, further comprising performing at least one of the following additional steps: prior placing a quantity of liquid from the sample in contact with at least one surface, at least one dry or liquid composition is applied to the at least one surface; and/or prior to heating the quantity of liquid on the at least one surface, at least one dry or liquid composition is applied to the at least one surface.

Embodiment K. The method according to any one of embodiments A-J, wherein the at least one dry or liquid substance comprises a solution of citric acid and sodium citrate or any buffer solution.

Embodiment L. The method according to any one of embodiments A-K, wherein the at least one surface has an affinity for phospholipids and/or other analytes.

Embodiment M. The method according to embodiments A-L, wherein the at least one surface comprises a metal, wherein the metal is optionally a steel, wherein the steel is optionally a stainless steel, or optionally comprises passivated, pickled, or electropolished stainless steel.

Embodiment N. The method according to embodiment D, wherein the at least one analytical instrument comprises a mass spectrometer, and wherein the mass spectrometer optionally is in a negative ionization mode, and the at least one spectrum is collected with a lowest m/z of less than 100, about 100, about 300, about 400, about 600, about 700, about 800, about 1000, or greater than 1000 m/z and a highest m/z of less than 800, about 800, about 1000, about 1200, about 1500, about 1800, about 2000, about 2200, about 2400, about 2500 or greater than 2500 m/z.

Embodiment O. The method according to embodiment D, wherein the at least one analytical instrument comprises a MALDI mass spectrometer; wherein the MALDI mass spectrometer is in a negative ionization mode; and wherein the MALDI matrix is between about 0.5 μL and 2 μL or about 1 μL of a solution comprising an about 12:6:1 ratio mixture of chloroform:methanol:water to which about 10 mg/mL of beta-carboline has been added.

Embodiment P. The method according to embodiment B, wherein the determining or the estimating the presence, quantity, taxon, or other property of the at least one microbial organism in the sample using the at least one spectrum produced by the at least one analytical instrument comprises any one of: (a) comparing via a dot product or other metric the at least one spectrum to one or more reference spectra, wherein the reference spectra is in a library or database; (b) comparing via a dot product or other metric the at least one spectrum to one or more averaged, summation, or synthetic spectra, wherein the averaged, summation, or synthetic spectrum is in a library or database; (c) classifying the at least one spectrum or estimating a value for the at least one spectrum with a support vector machine, a support vector machine ensemble, a neural network, a random forest, or any other machine learning device; or (d) any combination thereof.

Embodiment Q. The method according to any one of embodiments A, B, E, I, J, L, or M, wherein the at least one surface comprises a substantially flat plate suitable to be a MALDI plate.

Embodiment R. A method for extracting phospholipids and/or other analytes from a sample containing outer membrane vesicles (OMVs), membrane vesicles (MVs), or a membrane, the method comprising:

placing a quantity of liquid from the sample containing OMVs, MVs, or a membrane in contact with at least one surface, wherein the at least one surface is enclosed to facilitate control of evaporation of the quantity of liquid;

heating the quantity of liquid on the at least one surface;

optionally washing the at least one surface;

optionally allowing the at least one surface to at least partially dry; and

optionally applying a composition comprising at least one solvent and an optional substance having the property of acting as a MALDI matrix.

Embodiment S. The method according to any one of embodiments A-R, further comprising determining and/or measuring a response of at least one of a cell, cell culture, tissue, microbial community, or biofilm; wherein the response indicates antimicrobial resistance and/or susceptibility; and/or wherein the response measures one or more of an absorption/administration, distribution, metabolism, excretion, and toxicity (ADME-Tox) parameter, efficacy, release profile, toxicity, toxicology, and/or drug treatment effect for at least one treatment composition.

Embodiment T. A kit for use in practicing a method according to any one of embodiments A-S, the kit comprising any one or more of:

an analytical instrument, optionally a spectrometer;

a plate, wherein the plate optionally comprises a material disposed on at least a portion of at least one surface of the plate, and/or comprises at least one structured spot, wherein the structured spot comprises a substantially flat surface comprising one or more of a pore, a depression, or a channel having two ends, of which only one of the ends is open;

a gasket, wherein the gasket comprises at least one hole having a shape and a size configured for placement over and alignment with at least one structured spot on the plate;

a solvent, wherein the solvent comprises any one or more of a buffer solution, an acidic solution, and a solid to which a liquid can be added to form a buffer or an acidic solution;

an enclosure, wherein the enclosure comprises a holder base, optionally the holder base comprises a reservoir, and a lid; and

a computing device or system comprising a software configured to receive and/or display spectroscopic data.

Embodiment U. The kit of embodiment T, wherein use of the kit comprises:

placing a quantity of a liquid from a sample comprising a microbial organism in contact with the at least one surface, wherein the at least one surface is enclosed to facilitate control of evaporation of the quantity of liquid;

heating the quantity of liquid on the at least one surface;

optionally washing the at least one surface;

optionally allowing the at least one surface to at least partially dry;

optionally applying a composition comprising at least one solvent and an optional substance having the property of acting as a MALDI matrix;

analyzing the contents of the at least one surface with the at least one analytical instrument;

determining or estimating the presence, quantity, taxon, or other property of the at least one microbial organism in the sample using at least one spectrum produced by the at least one analytical instrument;

wherein the taxon or the other property of the at least one microbial organism is a bacterial species; a yeast and/or fungal species; a microbial species other than bacteria, a yeast, or a fungi; a microbial strain that is resistant, intermediately resistant, or susceptible to one or more antimicrobials, classes of antimicrobials, combinations of antimicrobials, and/or combinations of classes of antimicrobials; and wherein the other property is a microbial taxonomic classification above the level of species; a microbial taxonomic classification below the level of species, or a Gram stain classification;

wherein the at least one analytical instrument comprises a mass spectrometer, optionally a MALDI mass spectrometer, a Raman spectrometer, or a spectroscopic instrument;

wherein the heating comprises heating the quantity of liquid in contact with the at least one surface to less than 95° C., about 95° C., about 100° C., about 110° C., about 121° C., about 125° C., about 130° C., about 135° C., or more than 135° C.;

wherein the quantity of liquid is heated for less than 15 minutes, about 15 minutes, about 20 minutes, about 25 minutes, about 30 minutes, or longer than 30 minutes;

wherein the quantity of liquid has a volume of less than about 0.5 μL, about 0.5 μL, about 1.0 μL, about 1.5 μL, about 2 μL, about 3 μL, about 4 μL, about 6 μL, about 8 μL, about 10 μL, about 20 μL, or greater than 10 μL

wherein the quantity of liquid contains microbial organisms at a concentration of greater than 10⁹ Colony Forming Units (CFU)/mL, about 10⁹ CFU/mL, about 10⁸ CFU/mL, about 10⁷ CFU/mL, about 10⁶ CFU/mL, about 10⁵ CFU/mL, about 10⁴ CFU/mL, about 10³ CFU/mL, about 10² CFU/mL, about 10 CFU/mL, about 1 CFU/mL, or less than 1 CFU/mL, or wherein the quantity of liquid contains no microbial organisms;

wherein the heating comprises heating the quantity of liquid is heated by any one of the following: heating the at least one surface, heating the atmosphere surrounding the quantity of liquid and/or the at least one surface, directly heating the quantity of liquid, and/or any combination of thereof;

further comprising performing at least one of the following additional steps: prior placing a quantity of liquid from the sample in contact with at least one surface, at least one dry or liquid composition is applied to the at least one surface; and/or prior to heating the quantity of liquid on the at least one surface, at least one dry or liquid composition is applied to the at least one surface;

wherein the at least one dry or liquid substance comprises a solution of citric acid and sodium citrate or any buffer solution; wherein the at least one surface has an affinity for phospholipids and/or other analytes;

wherein the at least one surface comprises a metal, wherein the metal is optionally a steel, wherein the steel is optionally a stainless steel, or optionally comprises passivated, pickled, or electropolished stainless steel;

wherein the at least one analytical instrument comprises a mass spectrometer, and wherein the mass spectrometer optionally is in a negative ionization mode, and the at least one spectrum is collected with a lowest m/z of less than 100, about 100, about 300, about 600, about 700, about 800, about 1000, or greater than 1000 m/z and a highest m/z of less than 800, about 800, about 1000, about 1200, about 1500, about 1800, about 2000, about 2200, about 2400, about 2500 or greater than 2500 m/z;

wherein the at least one analytical instrument comprises a MALDI mass spectrometer; wherein the MALDI mass spectrometer is in a negative ionization mode; and wherein the MALDI matrix is between about 0.5 μL and 2 μL or about 1 μL of a solution comprising an about 12:6:1 ratio mixture of chloroform:methanol:water to which about 10 mg/mL of beta-carboline has been added;

wherein the determining or the estimating the presence, quantity, taxon, or other property of the at least one microbial organism in the sample using the at least one spectrum produced by the at least one analytical instrument comprises any one of: (a) comparing via a dot product or other metric the at least one spectrum to one or more reference spectra, wherein the reference spectra is in a library or database; (b) comparing via a dot product or other metric the at least one spectrum to one or more averaged, summation, or synthetic spectra, wherein the averaged, summation, or synthetic spectrum is in a library or database; (c) classifying the at least one spectrum or estimating a value for the at least one spectrum with a support vector machine, a support vector machine ensemble, a neural network, a random forest, or any other machine learning device; or (d) any combination thereof; and

wherein the at least one surface comprises a substantially flat plate suitable to be a MALDI plate.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/750, 972, filed on Oct. 26, 2018, and U.S. Provisional Patent Application No. 62/796,247, filed on Jan. 24, 2019, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for extracting phospholipids and/or other analytes from a sample containing microbial organisms, the method comprising:
placing a quantity of liquid from the sample in contact with at least one surface, wherein the at least one surface is enclosed to facilitate control of evaporation of the quantity of liquid;
heating the quantity of liquid on the at least one surface;
optionally washing the at least one surface;
optionally allowing the at least one surface to at least partially dry; and
optionally applying a composition comprising at least one solvent and an optional substance having the property of acting as a MALDI matrix.

2. A method for detecting the presence of at least one microbial organism in a sample, the method comprising:
placing a quantity of liquid from the sample in contact with at least one surface, wherein the at least one surface is enclosed to facilitate control of evaporation of the quantity of liquid;
heating the quantity of liquid on the at least one surface;
optionally washing the at least one surface;
optionally allowing the at least one surface to at least partially dry;
optionally applying a composition comprising at least one solvent and an optional substance having the property of acting as a MALDI matrix;
analyzing the contents of the at least one surface with at least one analytical instrument; and
determining or estimating the presence, quantity, taxon, or other property of the at least one microbial organism in the sample using at least one spectrum produced by the at least one analytical instrument.

3. The method according to claim 2, wherein the taxon or the other property of the at least one microbial organism is a bacterial species; a yeast and/or fungal species; a microbial species other than bacteria, a yeast, or a fungi; a microbial strain that is resistant, intermediately resistant, or susceptible to one or more antimicrobials, classes of antimicrobials, combinations of antimicrobials, and/or combinations of classes of antimicrobials; and wherein the other property is a microbial taxonomic classification above the level of species; a microbial taxonomic classification below the level of species, or a Gram stain classification.

4. The method according to claim 2, wherein the at least one analytical instrument comprises a mass spectrometer, optionally a MALDI mass spectrometer, a Raman spectrometer, or a spectroscopic instrument.

5. The method according to any one of claims 1-4, wherein the heating comprises heating the quantity of liquid in contact with the at least one surface to less than 95° C., about 95° C., about 100° C., about 110° C., about 121° C., about 125° C., about 130° C., about 135° C., or more than 135° C.

6. The method according to any one of claims 1-5, wherein the quantity of liquid is heated for less than 15 minutes, about 15 minutes, about 20 minutes, about 25 minutes, about 30 minutes, or longer than 30 minutes.

7. The method according to any one of claims 1-6, wherein the quantity of liquid has a volume of less than about 0.5 μL, about 0.5 μL, about 1.0 μL, about 1.5 μL, about 2 μL, about 3 μL, about 4 μL, about 6 μL, about 8 μL, about 10 μL, about 20 μL, or greater than 10 μL.

8. The method according to any one of claims 1-7, wherein the quantity of liquid contains microbial organisms at a concentration of greater than 10⁹ Colony Forming Units (CFU)/mL, about 10⁹ CFU/mL, about 10⁸ CFU/mL, about 10⁷ CFU/mL, about 10⁶ CFU/mL, about 10⁵ CFU/mL, about 10⁴ CFU/mL, about 10³ CFU/mL, about 10² CFU/mL, about 10 CFU/mL, about 1 CFU/mL, or less than 1 CFU/mL, or wherein the quantity of liquid contains no microbial organisms.

9. The method according to any one of claims 1-8, wherein the heating comprises heating the quantity of liquid by any one of the following: heating the at least one surface, heating the atmosphere surrounding the quantity of liquid , directly heating the quantity of liquid, and/or any combination thereof.

10. The method according to any one of claims 1-9, further comprising performing at least one of the following additional steps: prior placing a quantity of liquid from the sample in contact with at least one surface, at least one dry or liquid composition is applied to the at least one surface; and/or prior to heating the quantity of liquid on the at least one surface, at least one dry or liquid composition is applied to the at least one surface.

11. The method according to any one of claims 1-10, wherein the at least one dry or liquid substance comprises a solution of citric acid and sodium citrate or any buffer solution.

12. The method according to any one of claims 1-11, wherein the at least one surface has an affinity for phospholipids and/or other analytes.

13. The method according to any one of claims 1-12, wherein the at least one surface comprises a metal, wherein the metal is optionally a steel, wherein the steel is optionally a stainless steel, or optionally comprises passivated, pickled, or electropolished stainless steel.

14. The method according to any one of claims 4-13, wherein the at least one analytical instrument comprises a mass spectrometer, and wherein the mass spectrometer optionally is in a negative ionization mode, and the at least one spectrum is collected with a lowest m/z of less than 100, about 100, about 300, about 400, about 600, about 700, about 800, about 1000, or greater than 1000 m/z and a highest m/z of less than 800, about 800, about 1000, about 1200, about 1500, about 1800, about 2000, about 2200, about 2400, about 2500 or greater than 2500 m/z.

15. The method according to any one of claims 4-14, wherein the at least one analytical instrument comprises a MALDI mass spectrometer; wherein the MALDI mass spectrometer is in a negative ionization mode; and wherein the MALDI matrix is between about 0.5 μL and 2 μL or about 1 μL of a solution comprising an about 12:6:1 ratio mixture of chloroform:methanol:water to which about 10 mg/mL of beta-carboline has been added.

16. The method according to any one of claims 2-15, wherein the determining or the estimating the presence, quantity, taxon, or other property of the at least one microbial organism in the sample using the at least one spectrum produced by the at least one analytical instrument comprises any one of: (a) comparing via a dot product or other metric the at least one spectrum to one or more reference spectra, wherein the reference spectra is in a library or database; (b) comparing via a dot product or other metric the at least one spectrum to one or more averaged, summation, or synthetic spectra, wherein the averaged, summation, or synthetic spectrum is in a library or database; (c) classifying the at least one spectrum or estimating a value for the at least one spectrum with a support vector machine, a support vector machine ensemble, a neural network, a random forest, or any other machine learning device; or (d) any combination thereof.

17. The method according to any one of claim 1, 2, 5, 9, 10, 12, or 13, wherein the at least one surface comprises a substantially flat plate suitable to be a MALDI plate.

18. A method for extracting phospholipids and/or other analytes from a sample containing outer membrane vesicles (OMVs), membrane vesicles (MVs), or a membrane, the method comprising:
placing a quantity of liquid from the sample containing OMVs, MVs, or a membrane in contact with at least one surface, wherein the at least one surface is enclosed to facilitate control of evaporation of the quantity of liquid;
heating the quantity of liquid on the at least one surface;
optionally washing the at least one surface;
optionally allowing the at least one surface to at least partially dry; and
optionally applying a composition comprising at least one solvent and an optional substance having the property of acting as a MALDI matrix.

19. The method according to any one of claims 1-18, further comprising determining and/or measuring a response of at least one of a cell, cell culture, tissue, microbial community, or biofilm; wherein the response indicates antimicrobial resistance and/or susceptibility; and/or wherein the response measures one or more of an absorption/administration, distribution, metabolism, excretion, and toxicity (ADME-Tox) parameter, efficacy, release profile, toxicity, toxicology, and/or drug treatment effect for at least one treatment composition.

20. A kit for use in practicing a method according to any one of claims 1-19, the kit comprising any one or more of:
an analytical instrument, optionally a spectrometer;
a plate, wherein the plate optionally comprises a material disposed on at least a portion of at least one surface of the plate, and/or comprises at least one structured spot, wherein the structured spot comprises a substantially flat surface comprising one or more of a pore, a depression, or a channel having two ends, of which only one of the ends is open;
a gasket, wherein the gasket comprises at least one hole having a shape and a size configured for placement over and alignment with at least one structured spot on the plate;
a solvent, wherein the solvent comprises any one or more of a buffer solution, an acidic solution, and a solid to which a liquid can be added to form a buffer or an acidic solution;
an enclosure, wherein the enclosure comprises a holder base, optionally the holder base comprises a reservoir, and a lid; and
a computing device or system comprising a software configured to receive and/or display spectroscopic data.

21. The kit of claim 20, wherein use of the kit comprises:
placing a quantity of a liquid from a sample comprising a microbial organism in contact with the at least one surface, wherein the at least one surface is enclosed to facilitate control of evaporation of the quantity of liquid;
heating the quantity of liquid on the at least one surface;
optionally washing the at least one surface;
optionally allowing the at least one surface to at least partially dry;
optionally applying a composition comprising at least one solvent and an optional substance having the property of acting as a MALDI matrix;
analyzing the contents of the at least one surface with the at least one analytical instrument;
determining or estimating the presence, quantity, taxon, or other property of the at least one microbial organism in the sample using at least one spectrum produced by the at least one analytical instrument;
wherein the taxon or the other property of the at least one microbial organism is a bacterial species; a yeast and/or fungal species; a microbial species other than bacteria, a yeast, or a fungi; a microbial strain that is resistant, intermediately resistant, or susceptible to one or more antimicrobials, classes of antimicrobials, combinations of antimicrobials, and/or combinations of classes of antimicrobials; and wherein the other property is a microbial taxonomic classification above the level of species; a microbial taxonomic classification below the level of species, or a Gram stain classification;
wherein the at least one analytical instrument comprises a mass spectrometer, optionally a MALDI mass spectrometer, a Raman spectrometer, or a spectroscopic instrument;
wherein the heating comprises heating the quantity of liquid in contact with the at least one surface to less than 95° C., about 95° C., about 100° C., about 110° C., about 121° C., about 125° C., about 130° C., about 135° C., or more than 135° C.;
wherein the quantity of liquid is heated for less than 15 minutes, about 15 minutes, about 20 minutes, about 25 minutes, about 30 minutes, or longer than 30 minutes;
wherein the quantity of liquid has a volume of less than about 0.5 μL, about 0.5 μL, about 1.0 μL, about 1.5 μL, about 2 μL, about 3 μL, about 4 μL, about 6 μL, about 8 μL, about 10 μL, about 20 μL, or greater than 10 μL
wherein the quantity of liquid contains microbial organisms at a concentration of greater than 10⁹ Colony Forming Units (CFU)/mL, about 10⁹ CFU/mL, about 10⁸ CFU/mL, about 10⁷ CFU/mL, about 10⁶ CFU/mL, about 10⁵ CFU/mL, about 10⁴ CFU/mL, about 10³ CFU/mL, about 10² CFU/mL, about 10 CFU/mL, about 1 CFU/mL, or less than 1 CFU/mL, or wherein the quantity of liquid contains no microbial organisms;
wherein the heating comprises heating the quantity of liquid is heated by any one of the following: heating the at least one surface, heating the atmosphere surrounding the quantity of liquid and/or the at least one surface, directly heating the quantity of liquid, and/or any combination of thereof;
further comprising performing at least one of the following additional steps:
prior placing a quantity of liquid from the sample in contact with at least one surface, at least one dry or liquid composition is applied to the at least one surface; and/or prior to heating the quantity of liquid on the at least one surface, at least one dry or liquid composition is applied to the at least one surface;
wherein the at least one dry or liquid substance comprises a solution of citric acid and sodium citrate or any buffer solution;
wherein the at least one surface has an affinity for phospholipids and/or other analytes;
wherein the at least one surface comprises a metal, wherein the metal is optionally a steel, wherein the steel is optionally a stainless steel, or optionally comprises passivated, pickled, or electropolished stainless steel;
wherein the at least one analytical instrument comprises a mass spectrometer, and wherein the mass spectrometer optionally is in a negative ionization mode, and the at least one spectrum is collected with a lowest m/z of less than 100, about 100, about 300, about 600, about 700, about 800, about 1000, or greater than 1000 m/z and a highest m/z of less than 800, about 800, about 1000, about 1200, about 1500, about 1800, about 2000, about 2200, about 2400, about 2500 or greater than 2500 m/z;
wherein the at least one analytical instrument comprises a MALDI mass spectrometer; wherein the MALDI mass spectrometer is in a negative ionization mode; and wherein the MALDI matrix is between about 0.5 μL and 2 μL or about 1 μL of a solution comprising an about 12:6:1 ratio mixture of chloroform:methanol:water to which about 10 mg/mL of beta-carboline has been added;
wherein the determining or the estimating the presence, quantity, taxon, or other property of the at least one microbial organism in the sample using the at least one spectrum produced by the at least one analytical instrument comprises any one of: (a) comparing via a dot product or other metric the at least one spectrum to one or more reference spectra, wherein the reference spectra is in a library or database; (b) comparing via a dot product or other metric the at least one spectrum to one or more averaged, summation, or synthetic spectra, wherein the averaged, summation, or synthetic spectrum is in a library or database; (c) classifying the at least one spectrum or estimating a value for the at least one spectrum with a support vector machine, a support vector machine ensemble, a neural network, a random forest, or any other machine learning device; or (d) any combination thereof; and
wherein the at least one surface comprises a substantially flat plate suitable to be a MALDI plate.
